(19) United States
(12) Patent Application Publication
(10) Pub. No.: US 2021/0227311 A1
(43) Pub. Date: Jul. 22, 2021

(54) EAR-WORN DEVICE

(71) Applicant: Human, Incorporated, Seattle, WA (US)

(72) Inventors: Stephen John Minarsch, Seattle, WA (US); Lea Martin, Seattle, WA (US); Adam Abraham Jossem, Seattle, WA (US); Dae Il Kwon, Seattle, WA (US); Francesca Alyssum Quaglia, Seattle, WA (US); Benjamin S. Willis, Bellevue, WA (US); Joseph B. Moak, San Carlos, CA (US); Sam Rhea Sarcia, Lakeside, CA (US); Donovan Deame, San Francisco, CA (US); Douglas B. Satzger, Menlo Park, CA (US); Yuri Litvinov, San Francisco, CA (US); Faisal Mohammed Alshaker, San Francisco, CA (US); Ichiro Yamada, Issaquah, WA (US); Kellen H. Chow, Bellevue, WA (US); Insang Hwang, Buffalo Grove, WA (US); Ferdinand Ygona Laurino, Seattle, WA (US)

(21) Appl. No.: 16/956,444

(22) PCT Filed: Dec. 19, 2018
(86) PCT No.: PCT/US18/66621

§ 371 Date: Jun. 19, 2020

Related U.S. Application Data
(60) Provisional application No. 62/607,867, filed on Dec. 19, 2017.
(60) Provisional application No. 62/741,134, filed on Oct. 4, 2018.

(51) Int. Cl. H04R 1/10; H04R 5/033

(52) U.S. Cl. CPC H04R 1/105; H04R 1/1025; H04R 1/1066; H04R 1/1008; H04R 1/1041; H04R 1/1083; H04R 5/0335

(57) ABSTRACT

Various embodiments provide for an improved ear-worn device for attaching the ear-worn device to an ear of a user using a hooking body. The hooking body may be secured to the user's ear. In some embodiments, a device body may be coupled to the hooking body via a hinge. The device body may be moved (relative to the hooking body) using the hinge to cause the ear-worn device to transition from a closed configuration to an open configuration, and vice versa.

### RELATED APPLICATIONS

This application claims the benefit of priority to U.S. Provisional Application No. 62/607,867 entitled “EAR-WORN DEVICE,” filed on Dec. 19, 2017, and U.S. Provisional Application No. 62/741,134 entitled “EAR-WORN DEVICE,” filed on Oct. 4, 2018, the entire contents of the foregoing hereby incorporated by reference.

### BACKGROUND

Users of wearable audio systems—such as headphones and earphones—can choose between several different styles. However, current wearable audio systems typically utilize one of only a few common attachment mechanisms to secure these systems to the user. For example, one type of wearable audio system can be attached to a user's head via a headband that maintains the position of speakers on either side of the user's head. Another type of wearable audio system can be attached to the user by inserting the system into the user's ear canal. Current attachment mechanisms, including those described above, are often uncomfortable to wear, provide a suboptimal listening experience, or both. Further, such wearable audio systems typically compromise either battery life or mobility. For example, wearable audio systems inserted into users' ear canals typically offer relatively short battery life and high mobility, and other wearable audio system worn on a user's head via a headband typically provide longer battery life at the cost of reduced mobility As such, developing a wearable audio system that provide a user with a secure and comfortable attachment mechanism, a long battery life, a convenient form factor, and an excellent audio experience continues to be a design and technical challenge.

### SUMMARY

Various embodiments provide for an ear-worn device for coupling to an ear of a user. In some embodiments, the ear-worn device may include a device body, a hooking body, an ear pad couplable to the device body, and a hinge coupled to the device body and the hooking body. In such embodiments, the hinge may be configured to enable the device body to move towards and away from the hooking body between an open configuration and a closed configuration, the device body may include an edge member, the edge member may be configured to define a recessed area on the device body, and the ear pad may be positioned within the recessed area when coupled to the device body.

In some embodiments, the ear pad may include a stiffener member, a first padding member, and a second padding member that includes a posterior portion and an anterior portion. A front surface of the anterior portion of the second padding member may be coupled to a back surface of the first padding member, a front surface of the posterior portion of the second padding member may be coupled to a back surface of the stiffener member, and a front surface of the first padding member may be coupled to the back surface of the stiffener member. In some further embodiments, the first padding member may be configured to have a size substantially equal to a size of the anterior portion of the second padding member. Further, when the ear pad is coupled to the device body, the ear pad may protrude away from a surface of the recessed area by a first amount near an anterior side of the device body, the ear pad may protrude away from a surface of the recessed area by a second amount near a posterior side of the device body, and the first amount may be less than the second amount.

In some embodiments, the ear pad may include a stiffener member that includes at least one rigid material, a first padding member, and a second padding member that includes a posterior portion and an anterior portion. In such embodiments, a front surface of the posterior portion of the second padding member may be coupled to a back surface of the first padding member, a front surface of the anterior portion of the second padding member may be coupled to a back surface of the stiffener member, and a front surface of the first padding member may be coupled to the back surface of the stiffener member. Also, the first padding member may be configured to have a size substantially equal to a size of the posterior portion of the second padding member.

In some embodiments, the device body may include a first coupling device positioned towards a surface of the recessed area, the ear pad may include a second coupling device positioned towards a front surface of the ear pad, and the first coupling device may be coupled to the second coupling device when the ear pad is coupled to the device body. In some alternative or additional embodiments, the hooking body and the device body, when the ear-worn device is configured in an open configuration, may form a space suitable for receiving at least a posterior portion of a human ear. When at least the posterior portion of the human ear is positioned within the space formed by the hooking body and the device body, the hinge may be configured to urge the hooking body and the device body together so that the hooking body and the device body apply a compressive force to at least the posterior portion of the human ear.

In some embodiments, the ear-worn device may also include a ring member configured to have a shape at least substantially similar to an edge of the ear pad and to secure the ear pad to the device body within the recessed area. In some embodiments, the ear-worn device may also include an ear-fitting attachment having a profile shape that accommodates at least a shape of a root of a posterior portion of a human ear. In such embodiments, the profile shape may be substantially “C” shaped. In some alternative (or additional) embodiments, the ear-fitting attachment may be configured such that a cross-sectional shape of a first portion of an anterior surface of the ear-fitting attachment is substantially curved, a cross-sectional shape of a second portion of the anterior surface of the ear-fitting attachment is substantially flat, and the first portion of the anterior surface transitions into the second portion of the anterior surface. In some further embodiments, the ear-fitting attachment may be further configured such that a cross-sectional shape of a third portion of the anterior surface of the ear-fitting attachment is substantially curved, the second portion of the anterior surface transitions into the third portion of the anterior surface, and the first portion of the anterior surface is distinct from the third portion of the anterior surface. In some embodiments, the ear-fitting attachment may include a first coupling device, the hooking body may include a second coupling device, and the hooking body and the ear-fitting attachment may be couplable together by coupling the first coupling device of the ear-fitting attachment with the second coupling device of the hooking body. In some embodiments, when the hooking body and the ear-fitting attachment are coupled together, a surface of the hooking body may be contiguous with a surface of the ear-fitting attachment.

In some embodiments, the hinge may include a torsion spring that biases the device body and the hooking body towards each other. In some embodiments, while the ear-worn device is in a closed configuration, the hooking body and an engaging portion of the device body may be collectively configured to apply a compressive force to a posterior portion of the ear. In some embodiments, transitioning the ear-worn device from an open configuration to a closed configuration while the ear-worn device is secured to the ear may cause the ear pad to move proximate to an interior portion of the ear.

In some embodiments, the ear pad may include an acoustically transparent material, and the device body may further include a speaker system positioned beneath the ear pad and configured to output sound towards a back side of the device body through the ear pad. In such embodiments, the device body may further include a supplemental speaker system configured to output sound towards a front side of the device body.

In some embodiments, the device body may include a device cover positioned towards a front of the device body. The device cover may include at least one microphone port positioned at an anterior side of the device body and at least one microphone port positioned at a posterior side of the device body. In some embodiments, the device body may include a touch sensor positioned to a back of the device cover, and the device cover may include at least one material configured to allow the touch sensor to detect a touch input received on the device cover.

In some embodiments, the device body may include a first microphone positioned near a back side of the device body and near a speaker system, a second microphone positioned near a front, anterior side of the device body, a third microphone positioned near a front, anterior side of the device body. In such embodiments, the third microphone may be positioned closer to a bottom of the device body than the second microphone, and a fourth microphone may be positioned near a front, posterior side of the device body. In such embodiments, the ear-worn device may be configured to capture first sound from at least the third microphone and second sound from the fourth microphone, and the ear-worn device may be configured to perform beam-forming noise cancellation using the first sound and the second sound.

In some embodiments, the ear pad may include a pliable material configured to form at least a partial acoustic seal around an interior portion of a human ear when the ear-worn device is secured to the human ear. In some embodiments, the ear-worn device may include a processing unit, memory, and a sensor. The sensor may be configured to detect whether another ear-worn device is coupled to the ear-worn device, and the memory may include instructions that when executed by the processing unit cause the processing unit to receive a signal from the sensor indicating whether the other ear-worn device is coupled, selectively activate or resume at least one operation or function in response to determining that the other ear-worn device is not coupled to the ear-worn device, and selectively deactivate or suspend at least one operation or function in response to determining that the other ear-worn device is coupled to the ear-worn device.

Various embodiments provide for an ear-worn device for coupling to an ear of a user, and the ear-worn device may include a device body that includes an electrical component, a hooking body that includes a power source, and an electrical lead. In such embodiments, a first portion of the device body and a second portion of the hooking body may be configured to form a hinge, the hinge may be configured to enable the device body to move towards and away from the hooking body between an open configuration and a closed configuration, the electrical lead electrically may couple the electrical component to the power source, and the electrical lead may pass through the hinge.

In some embodiments, the hooking body may further include a charging connector electrically coupled to the power source. The electrical lead may pass, at least partially, through the hinge along an axis of rotation of the hinge. In some further embodiments, a portion of the electrical lead passing through the hinge may experience no greater than 15 degrees of rotation when the ear-worn device transitions from a closed configuration to an open configuration.

In some embodiments, the ear-worn device may further include a spring coupled to the first portion and the second portion. The spring may be configured to exert torque on the first portion and the second portion when coupled to the first portion and the second portion, the torque may cause the hooking body and the device body to remain physically engaged when the ear-worn device is configured in a closed configuration, and the torque may cause the hooking body and the device body to move together when the ear-worn device is configured in an open configuration. In some further embodiments, the device body may include a curved channel, and the spring may be positioned in the curved channel when the spring is coupled to the first portion and the second portion. In some embodiments, the spring may be coupled to the first portion via a first spring anchor, and the spring may be coupled to the second portion via a second spring anchor.

In some embodiments, the hooking body may include a first device coupling element, a second device coupling element, a first charger coupling element, a second charger coupling element, a power charging connector, and a battery as the power source. The first device coupling element and the second device coupling element may be configured to secure the ear-worn device to another ear-worn device by coupling to a third device coupling element and a fourth device coupling element, respectively, included in the other ear-worn device. In some further embodiments, the first charger coupling element and the second charger coupling element may be configured to secure the ear-worn device to a charging device by coupling to a third charger coupling element and a fourth charger coupling element, respectively, included in the charging device.

In some embodiments, the battery may be configured to have an at least partially curved shape that at least partially conforms to a shape of a portion of the hooking body facing an anterior side of the ear-worn device. In such embodiments, the hooking body may include a first protruding portion that forms a cavity that protrudes from the hooking body towards an anterior side of the ear-worn device, and the battery may include a second protruding portion that occupies the cavity.

Various embodiments provide for an ear-worn device for coupling to an ear of a user. In such embodiments, the ear-worn device may include a device body that includes a speaker system and an acoustic barrier, a hooking body that includes a power source, an electrical lead, a device cover, and a hinge. The hinge may be configured to enable the device body and the hooking body to move towards and away from each other between an open configuration and a closed configuration, the electrical lead may electrically couple the speaker system to the power source, the electrical lead may pass through the hinge and the acoustic barrier, and the acoustic barrier and the device cover may be collectively configured to form, at least partially, an acoustically isolated volume of air around the speaker system.

In some embodiments, the device body may further include a supplemental speaker system, and the speaker system may be acoustically isolated from the supplemental speaker system. In some further embodiments, the device body may include a coupling channel, the acoustic barrier may include a portion of the coupling channel, the acoustic barrier and the device cover may be collectively configured to form, at least partially, the acoustically isolated volume of air around the speaker system by coupling a coupling device of the device cover to the coupling channel. In some embodiments, the device body may further include a speaker housing, and the speaker housing may accommodate the supplemental speaker system.

Various embodiments provide for an ear-worn device configured as described above and further configured as a personal listening device. Various embodiments provide for a system that includes an ear-worn device as described above. Various embodiments provide for a system that includes a first ear-worn device configured as described above and a second ear-worn device configured as a mirror-image of the first ear-worn device. In such embodiments, the first ear-worn device may include a first coupling device, the second ear-worn device may include a second coupling device, and the first coupling device and second coupling device may be configured to couple selectively together.

### BRIEF DESCRIPTION OF THE DRAWINGS

The foregoing embodiments and many of the attendant advantages will become more readily appreciated as the same become better understood by reference to the following detailed description, when taken in conjunction with the accompanying drawings, wherein:

FIGS. 1A-1H are exterior views of an ear-worn device, according to some embodiments.

FIGS. 2A-2E are exterior views of an ear-worn device secured to a user's ear, according to some embodiments.

FIGS. 3A-3L are exterior views of an ear-worn device having an alternative design, according to some embodiments.

FIGS. 4A-4J are partially transparent views of the ear-worn device depicted in FIGS. 3A-3L, according to some embodiments.

FIGS. 5A-5B are exploded views of the ear-worn device depicted in FIGS. 3A-4J, according to some embodiments.

FIG. 6A is an exploded view of an ear-pad and device body of an alternative ear-worn device, according to some embodiments.

FIG. 6B is a back-side partial view of an alternative ear-worn device that includes the device body depicted in FIGS. 3A-5B coupled to an ear pad having an alternative design, according to some embodiments.

FIG. 6C is a front-side view of the ear pad depicted in FIG. 6B, according to some embodiments.

FIGS. 6D-6E are exploded views of the ear pad depicted in FIGS. 6A-6C, according to some embodiments.

FIG. 6F is a cross-sectional view of the ear pad depicted in FIGS. 6B-6E, according to some embodiments.

FIG. 6G is a cross-sectional view of an ear pad having an alternative design, according to some embodiments.

FIG. 7A is an exterior view of a hooking body and sizing attachment of an alternative ear-worn device, according to some embodiments.

FIG. 7B is a cross-sectional view of the sizing attachment depicted in FIG. 7A, according to some embodiments.

FIGS. 7C-7D are external views of a hooking body and a sizing attachment having an alternative design, according to some embodiments.

FIGS. 7E-7H are additional external views of the sizing attachment depicted in FIGS. 7C-7D, according to some embodiments.

FIGS. 7I-7K are cross-sectional views of the sizing attachment depicted in FIGS. 7C-7H, according to some embodiments.

FIGS. 8A-8B are exterior views of an audio system that includes a first ear-worn device and a second ear-worn device, according to some embodiments.

FIG. 9 is a communication system diagram illustrating an ear-worn device configured as a wearable audio system, according to some embodiments.

### DETAILED DESCRIPTION

Various embodiments of the ear-worn device may be described with reference to certain anatomical features of a human user's ear. For ease of reference, the anatomical features of a user's ear may be referred to in this disclosure using the following terms. The term “root of an ear” refers to a portion of the ear that is proximal to the user's head. Specifically, the root of a user's ear may be a portion or structure of the ear that secures the ear to the user's head. Also, as used herein, the term “outer ear” refers to the portion of the ear that is distal to the user's head as compared to the root of the ear. The outer ear may include or otherwise be defined by at least a portion of the ear's auricle, helix, and/or lobule. Typically, the perimeter of the outer ear of an ear is greater than the perimeter of the root of the ear. The term “upper portion of the ear” generally refers to a portion of the ear that is proximal to the top of the user's head. In contrast, the term “lower portion of the ear” refers to a portion of the ear that is distal to the top of the user's head. Further, the term “anterior portion of an ear” refers to a portion of the ear that is proximal to a user's face and distal to the back of the user's head. The anterior portion of the ear may include portions of the helix, the antihelix, tragus, and antitragus that are proximal to the user's face. The term “posterior portion of an ear” refers to a portion of the outer ear that is proximal to the back of the user's head and distal to the user's face. The posterior portion of the ear may include portions of the helix and the antihelix proximal to the back of the user's head. The term “interior portion of an ear” refers to a portion of the outer ear proximal to, but not including, the ear canal. The interior portion of an ear may include, without limitation, at least part of one or more of the concha, anti-helix, anti-tragus, and tragus. Further descriptions and references to the foregoing terms are provided herein.

As generally described above, current attachment mechanisms used to secure wearable audio systems to users are often suboptimal. For example, some wearable audio systems implement a type of attachment mechanism commonly referred to as an “over-the-ear” design. A wearable audio system that utilizes an over-the-ear design is worn by a user by placing ear cups of the wearable audio system over and around the ears of the user. The ear cups are coupled to a headband positioned on the top or to the back of the user's head. The headband applies a compressive force to the user's head in order to secure the ear cups to the user, often resulting in headaches or general discomfort over time. This pressure is especially noticeable in some over-the-ear wearable audio systems that apply a substantial amount of pressure on the user to ensure that an acoustic seal is formed around the ear cups. Further, while over-the-ear wearable audio systems typically provide many hours of battery life, these systems often are bulky and difficult for users to transport easily (e.g., while commuting to or from work).

Other suboptimal wearable audio systems are also available. For example, some wearable audio systems are secured to users via insertion into users' ear canals. Often, such wearable audio systems cause users to experience discomfort over time by exerting a constant pressure against the ear canal walls. Further, while these systems can acoustically isolate the inner ear from external sounds, the seal is formed inside the ear canal, leading to an increased risk that audio played from such systems and directed into the ear canal will cause users to suffer hearing damage. Further, these “in-the-ear” style wearable audio systems typically have small form factors that, while relatively easy to transport, do not support large batteries. As such, these in-the-ear wearable audio systems typically have a reduced battery life that may be inadequate for some users.

In overview, aspects of the present disclosure include ear-worn devices and wearable audio systems that include attachment mechanisms that feature improvements over current attachment mechanisms, such as those described above. Specifically, according to various embodiments described herein, such ear-worn devices may enable users to secure the ear-worn devices to the users' ears more easily than currently available attachment mechanisms. Further, ear-worn devices described herein according to various embodiments may feature other aspects that result in an overall improved degree of mobility, battery life, security, and/or comfort when compared to conventional wearable audio systems.

In some embodiments, the ear-worn device may include a device body that is coupled to a hooking body. The device body may include various components (e.g., one or more speaker systems). The hooking body may include a power source (e.g., a battery) that is electronically coupled to one or more components included in the device body. The hooking body may be hooked behind a user's ear in order to secure the ear-worn device to the user's ear. Because the power source is included in the hooking body, the ear-worn device according to various embodiments may be more balanced and/or more comfortable than conventional wearable audio systems because the weight of the hooking body and the power source balances (at least somewhat) with the weight of the device body and the components included in the device body.

In some embodiments, the hooking body may secure the ear-worn device to at least a root of the upper portion of the user's ear and a root of a posterior portion of the user's ear. The hooking body may be coupled to a device body via a joint (e.g., a hinge or the like) that limits the range of movement of the device body in relation to the hooking body. While the hooking body is secured to the user's ear, the ear-worn device may be configured in an open configuration in which the device body is rotated or otherwise positioned away from the hooking body, and in some instances, may be biased away from engagement with the hooking body. The ear-worn device may transition from an open configuration to a closed configuration in which the device body physically engages the hooking body by an external force or a biasing member (e.g., a torsional spring included in the joint). While the ear-worn device transitions from an open configuration to a closed configuration, the device body (and, in some embodiments, the hooking body) may apply a slight compressive force on the posterior portion of the user's ear to secure the ear-worn device to the ear.

As described, the device body may be coupled to the hooking body via a joint. As used herein, the term “joint” may generally refer to a mechanism or device that couples the device body to the hooking body and that enables the device body to move (e.g., pivot, rotate) in a direction towards and a direction away from engagement with the hooking body. For example, a joint may include a pin hinge, a ball-and-socket joint, a tension hinge, other mechanical hinge, or one of various other types of devices configured to movably couple the hooking body and the device body together and configured to limit the movement of the device body in relation to the hooking body. In some embodiments, the joint may be configured to enable the ear-worn device to transition between an open configuration and a closed configuration. When the ear-worn device is in an open configuration, the device body is moved away from engagement with the hooking body. For example, the device body may receive an external force that causes the device body to swing away from the hooking body about the joint. When the ear-worn device is in a closed configuration, the device body and the hooking body may physically engage or, if worn by the user, may collectively apply a compressive force on the posterior portion of the user's ear (e.g., as described). In some instances, a biasing member (e.g., torsional spring) may be provided to urge the ear-worn device into a closed configuration and, optionally, one or more locking features may be provided to hold the ear-worn device in the closed configuration (e.g., magnetic attraction between magnetic element(s) included in the hooking body and the device body). In such embodiments, a user may be required to overcome the force of the biasing member in order to transition the ear-worn device from a closed configuration to an open configuration.

In some optional embodiments, each of the hooking body and the device body may include one or more coupling devices. In such embodiments, one or more first coupling devices of the device body may be configured to engage one or more second coupling devices of the hooking body in order to fasten or otherwise secure the device body to the hooking body. Accordingly, in some embodiments, the ear-worn device may be deemed to be in a closed configuration when the one or more first coupling devices engage the one or more second coupling devices, and the ear-worn device may be deemed to be in an open configuration when the one or more first coupling devices are released from or are not otherwise engaged with the one or more second coupling devices.

According to some embodiments, the ear-worn device for an example left ear device may be secured to the ear of the user by configuring the ear-worn device in an open configuration, hooking the hooking body to the upper root portion of the user's ear, and rotating the hooking body clockwise until the second portion of the hooking body engages the posterior root portion of the user's ear. Once the second portion of the hooking body engages the posterior root portion of the user's ear, the hooking body may not be able to continue rotating clockwise around the user's ear. The ear-worn device may be transitioned to a closed configuration by moving (e.g., swinging) the device body towards engagement with the hooking body until the device body fastens to or otherwise engages the hooking body. Additionally (or alternatively), the ear-worn device may include a biasing member that may transition the ear-worn device to a closed configuration from an open configuration in the absence of an external force. The ear-worn device may be unsecured and removed from the user's ear by performing the inverse of the above steps. Specifically, the ear-worn device may be transitioned from a closed configuration to an open configuration by unfastening or disengaging the device body from the hooking body and moving (e.g., swinging) the device body away from engagement with the hooking body. The ear-worn device may then be removed from the user's ear in part by rotating the hooking body counterclockwise to disengage the first and/or second portions of the hooking body from the user's ear.

In some embodiments, the ear-worn device may be configured as a wearable audio system. In such embodiments, the device body may include one or more audio components. By way of non-limiting examples, the audio components may include a speaker system, memory, a processing unit (e.g., a digital signal processor or central processing unit), a transceiver configured to receive audio data from external computing devices, or the like. The device body may be coupled to the device body such that a speaker is positioned in proximity to an ear canal of the user's ear.

As used herein, references to an anterior side of an ear-worn device (or subpart or portion of that ear-worn device, including, for example, a hooking body or device body) refers to a side, surface, or portion of the ear-worn device that is facing or nearby a user's face while the user is wearing the ear-worn device. Similarly, references to a posterior side of an ear-worn device (or subunit or portion of that ear-worn device, including, for example, a hooking body or device body) refers to a side, surface, or portion of the ear-worn device that is facing or nearby a back of a user's head while the user is wearing the ear-worn device.

Various embodiments will be described in detail with reference to the accompanying drawings. Wherever possible, the same reference numbers will be used throughout the drawings to refer to the same or like parts. References made to examples and implementations are for illustrative purposes and are not intended to limit the scope of the invention or the claims.

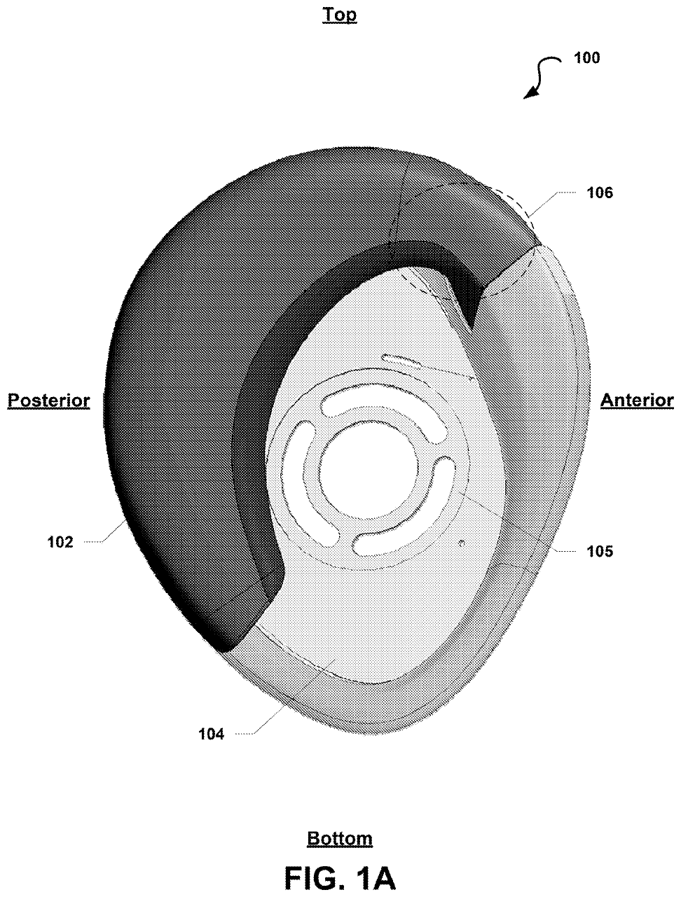

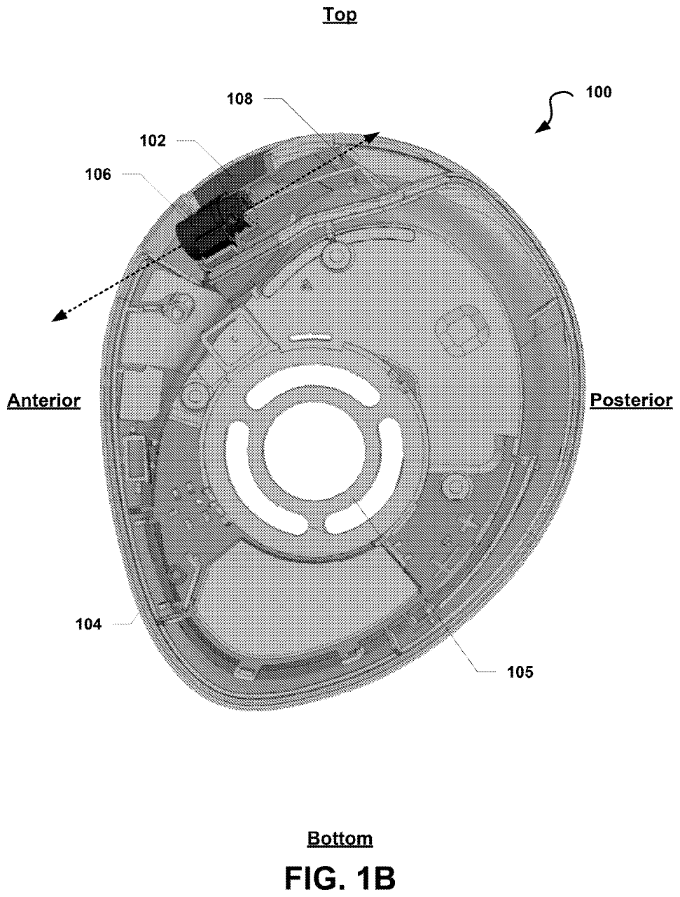

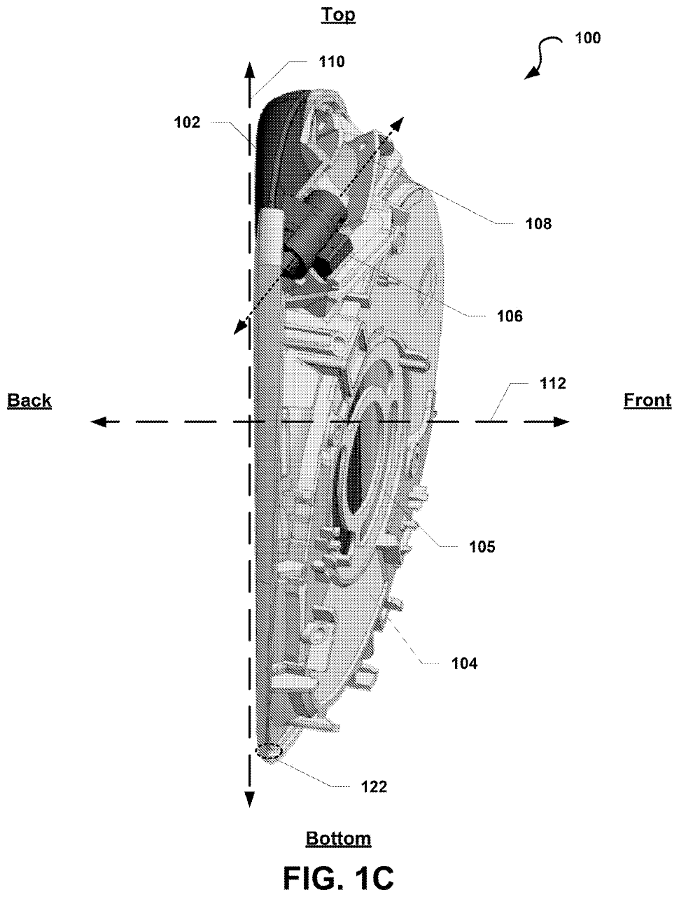

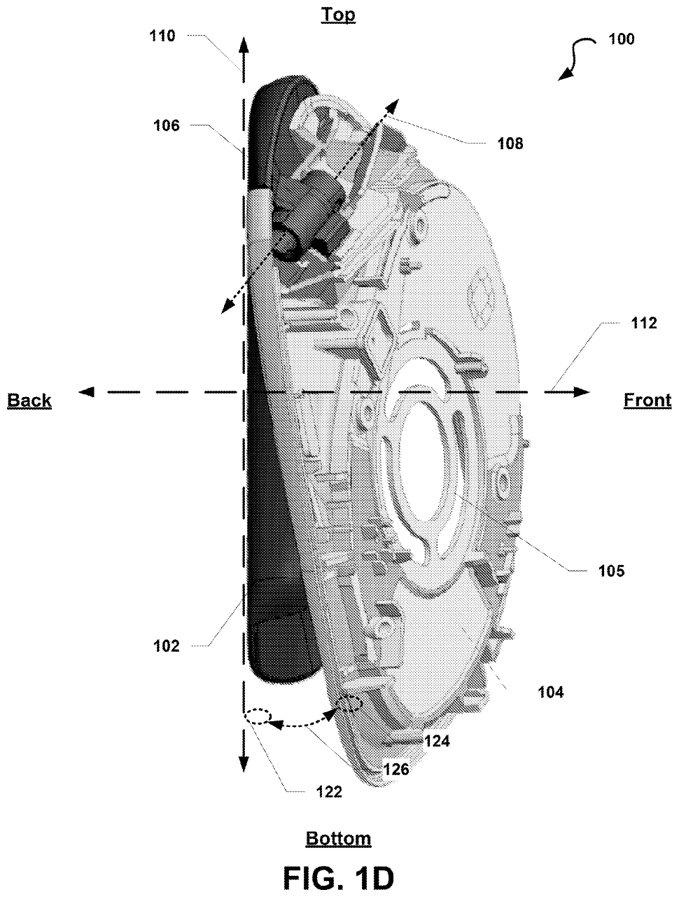

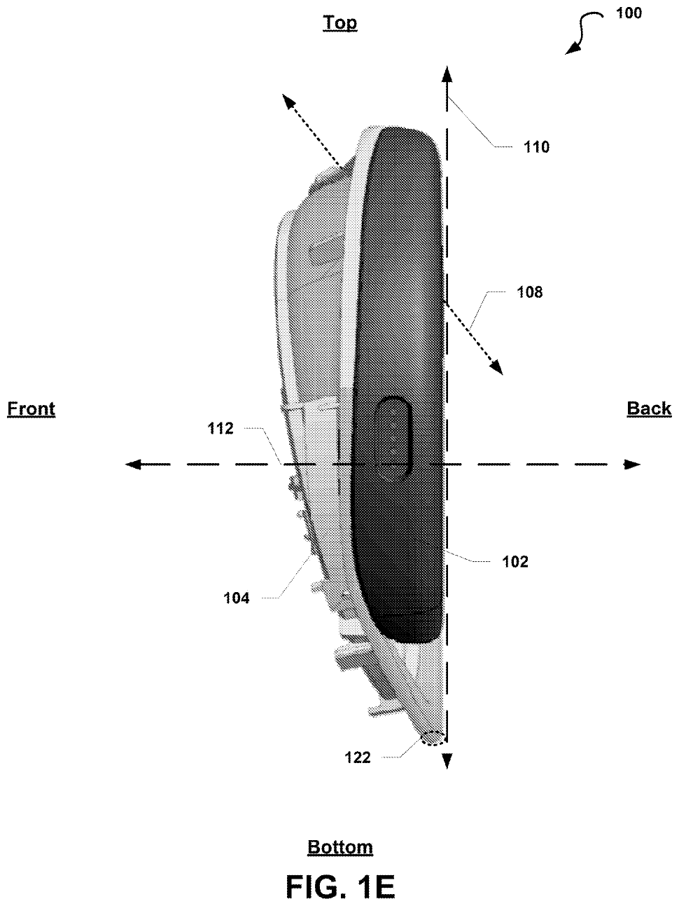

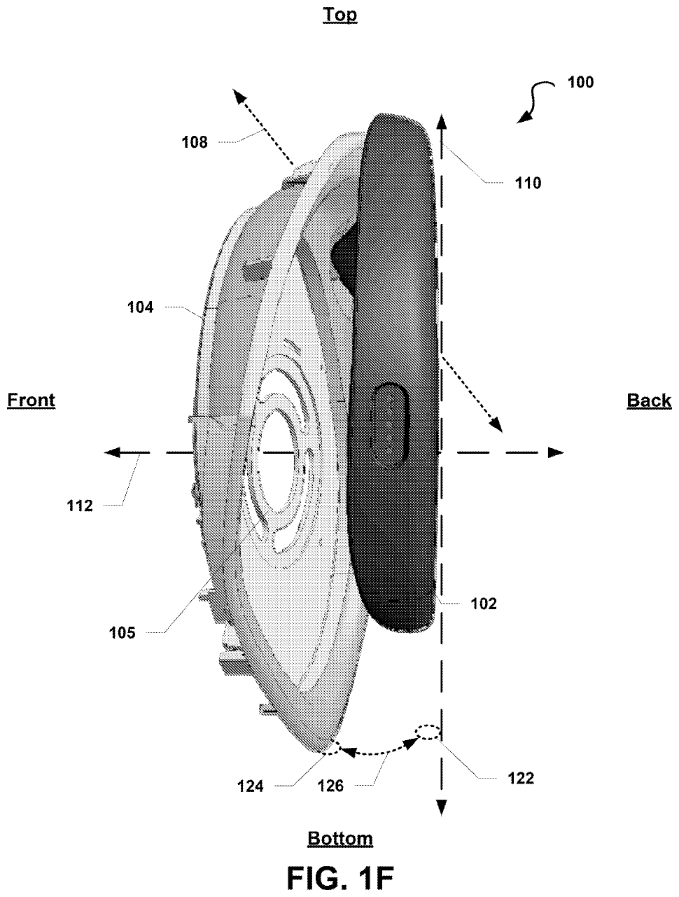

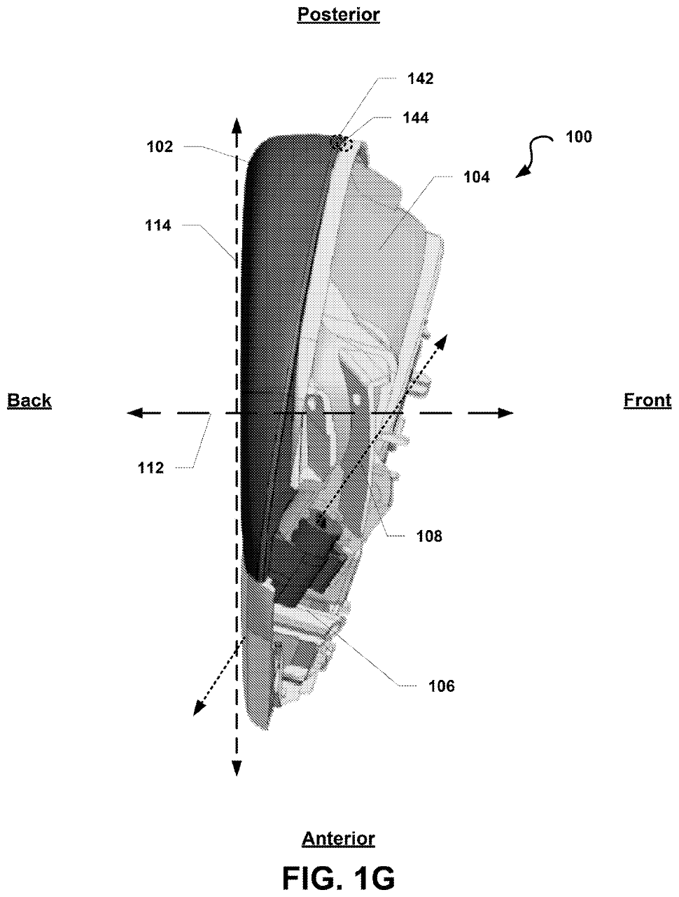

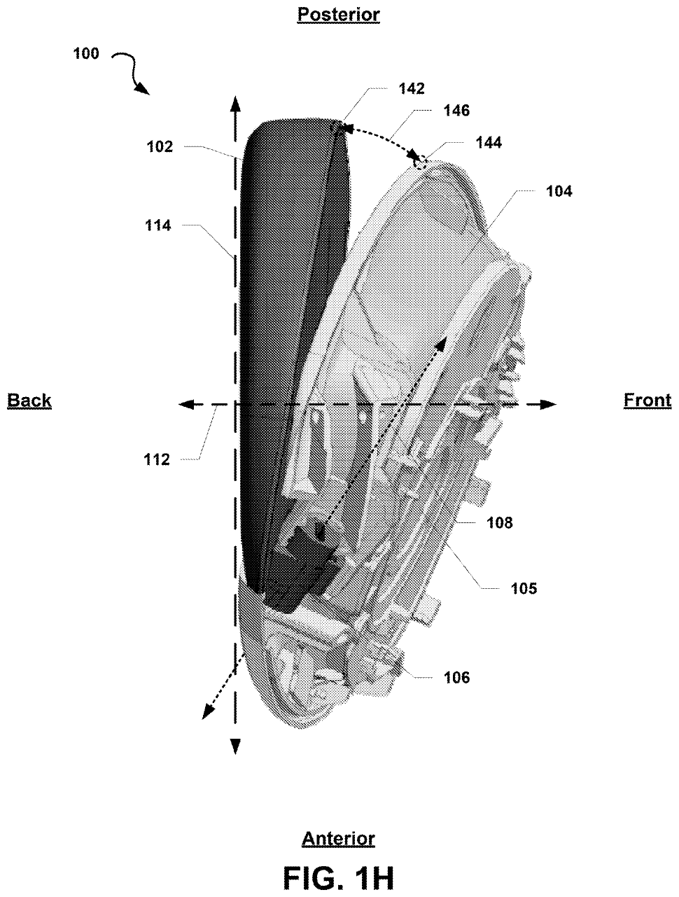

FIGS. 1A-1E illustrate exterior views of an ear-worn device 100, according to some embodiments. FIG. 1A illustrates an exterior view of a back of the ear-worn device 100 while the ear-worn device 100 is configured in a closed configuration. FIG. 1B illustrates an exterior view of a front side of the ear-worn device 100 while the ear-worn device 100 is configured in a closed configuration. FIG. 1C illustrates an exterior view of an anterior side of the ear-worn device 100 while the ear-worn device 100 is configured in a closed configuration. FIG. 1D illustrates an exterior view of an anterior side of the ear-worn device 100 while the ear-worn device 100 is configured in an open configuration. FIG. 1E illustrates an exterior view of a posterior side of the ear-worn device 100 while the ear-worn device 100 is configured in a closed configuration. FIG. 1F illustrates an exterior view of a posterior side of the ear-worn device 100 while the ear-worn device 100 is configured in an open configuration. FIG. 1G illustrates an exterior view of a top side of the ear-worn device 100 while the ear-worn device 100 is configured in a closed configuration. FIG. 1H illustrates an exterior view of a top side of the ear-worn device 100 while the ear-worn device 100 is configured in an open configuration.

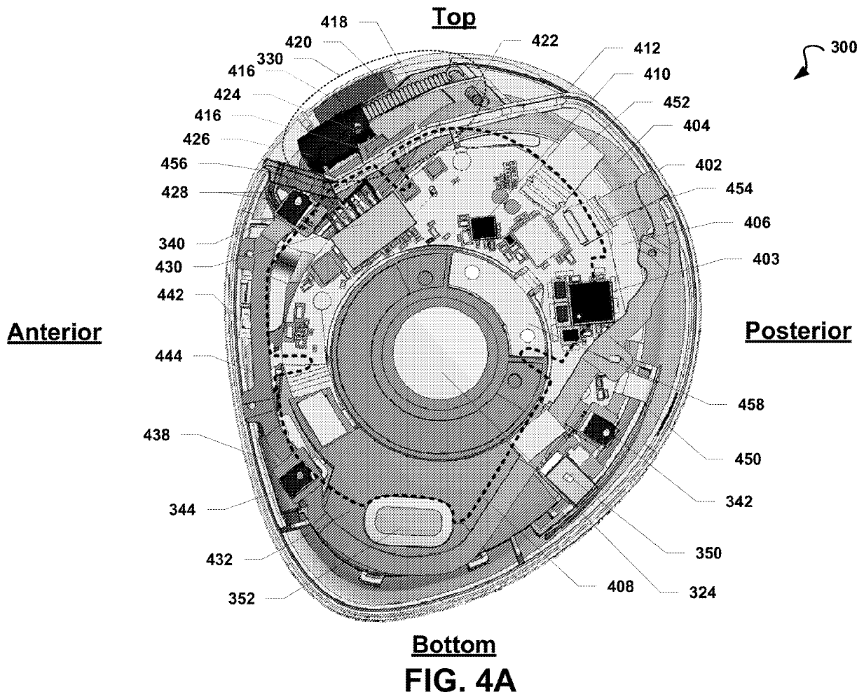

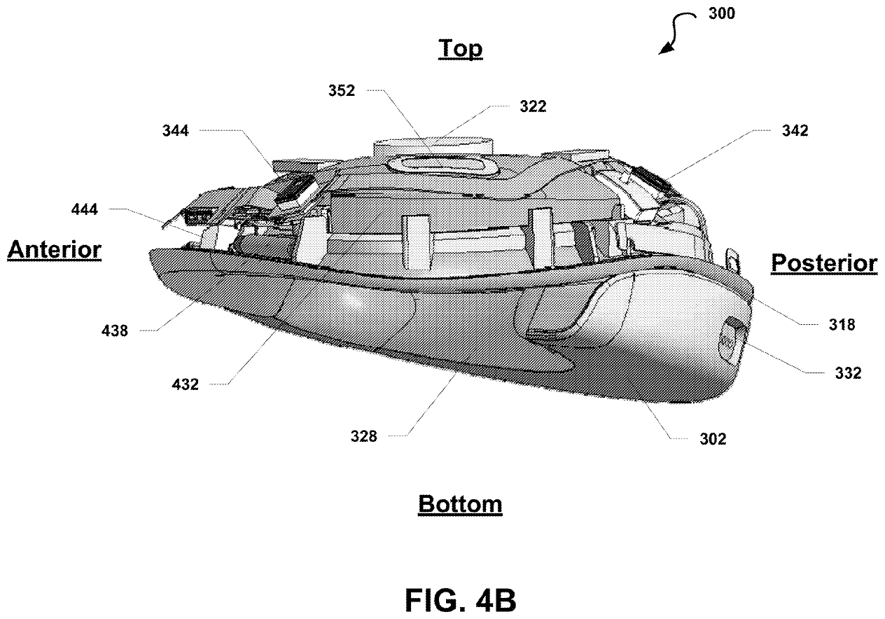

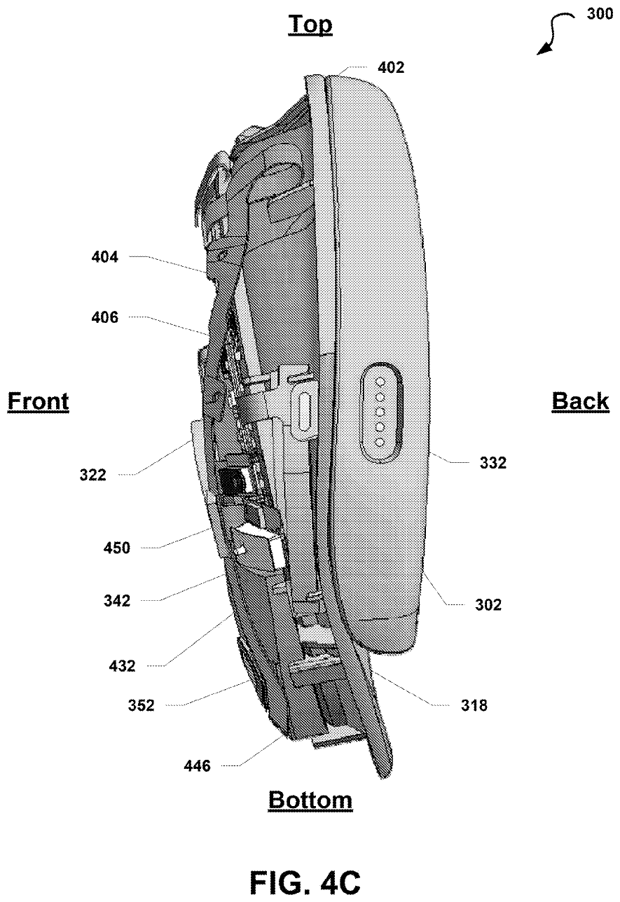

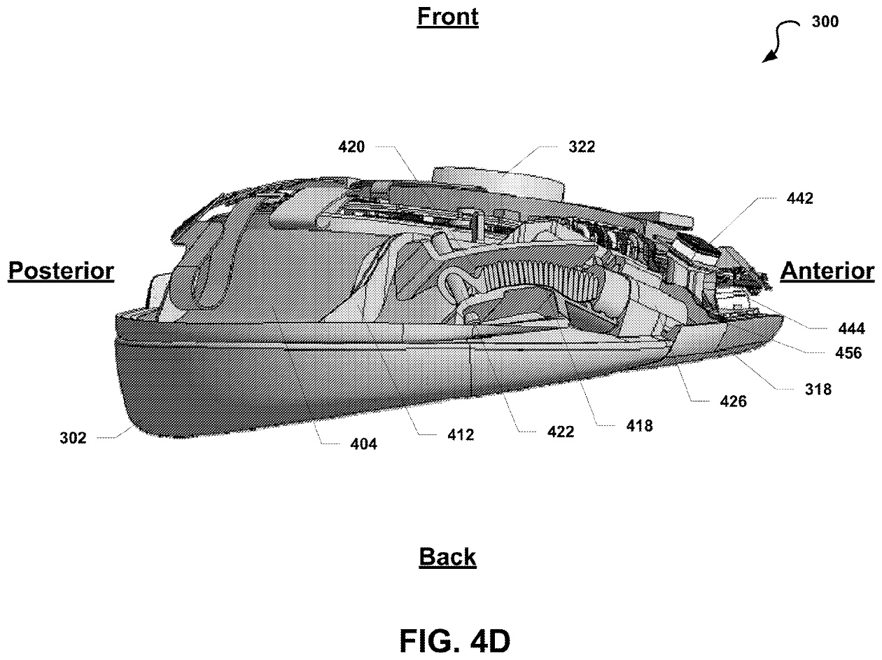

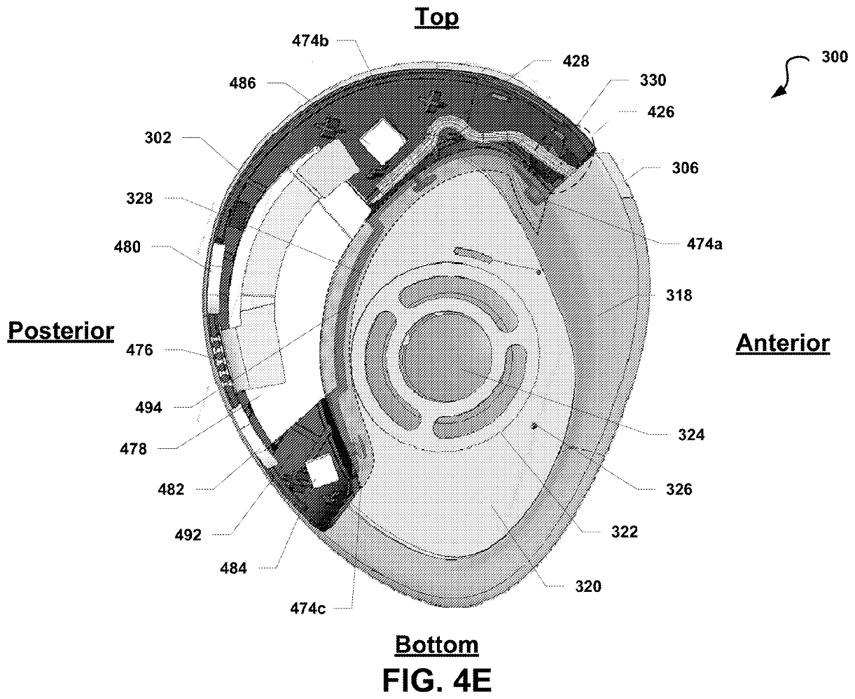

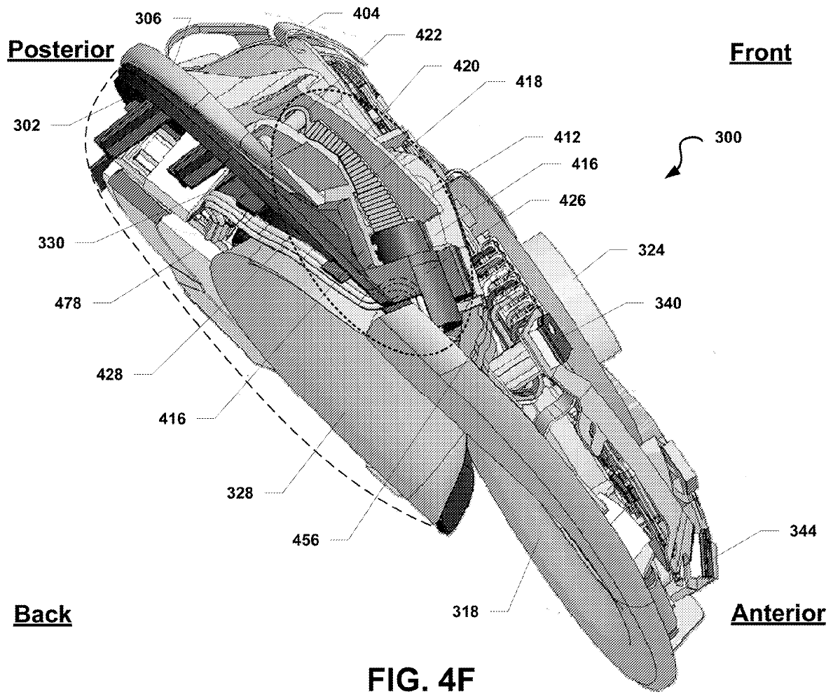

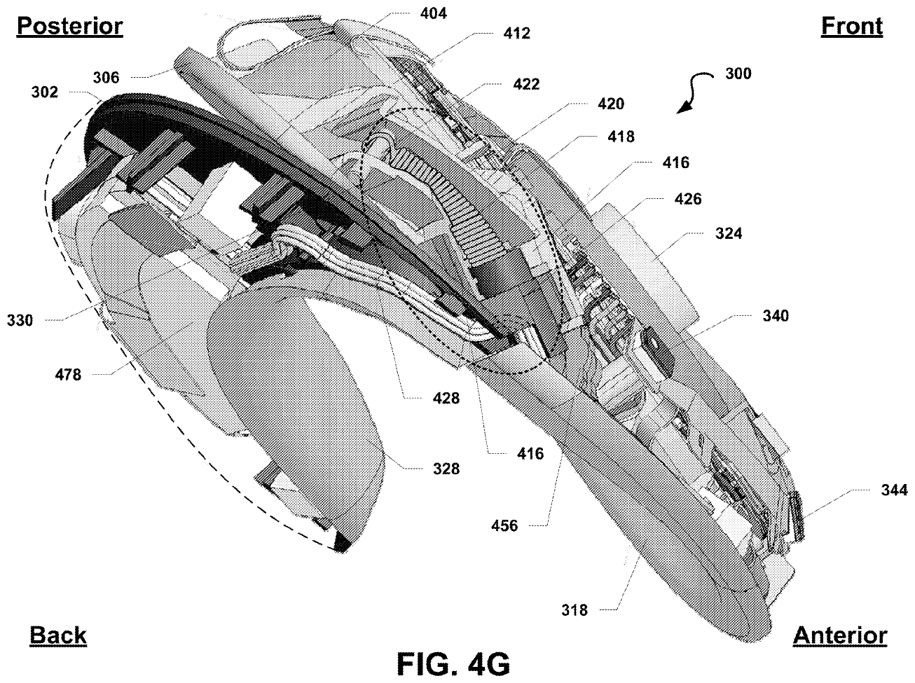

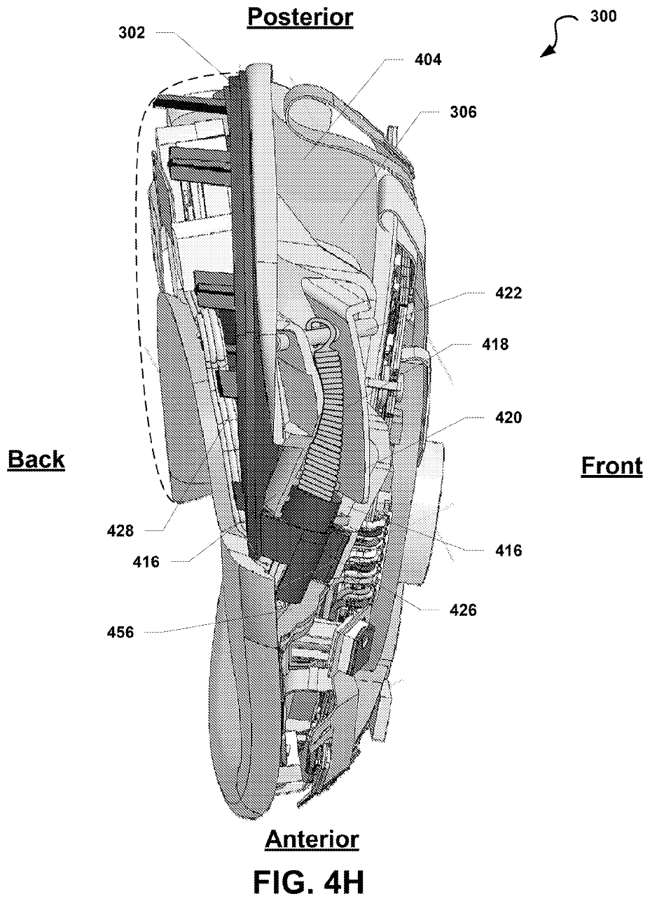

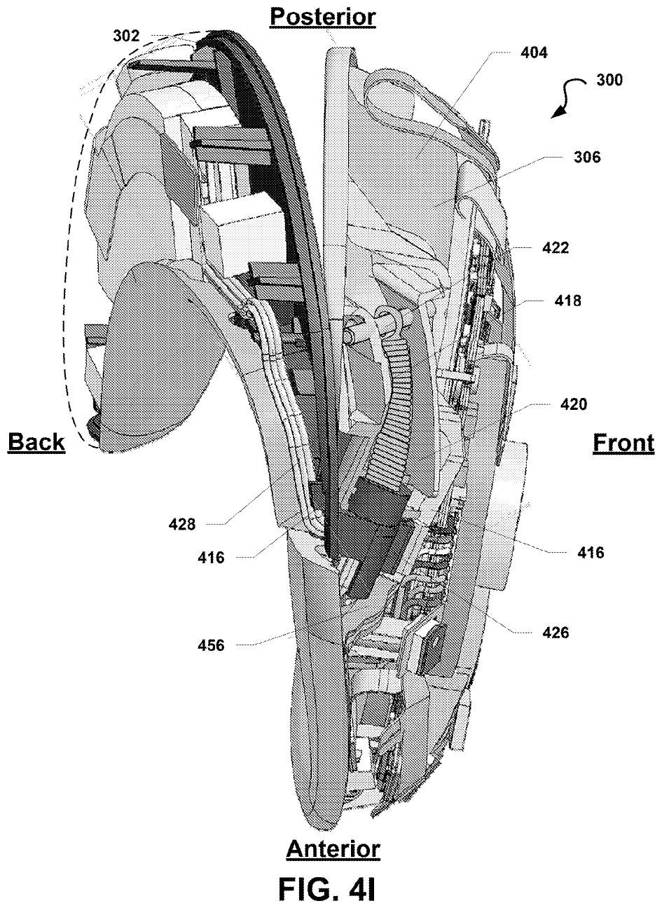

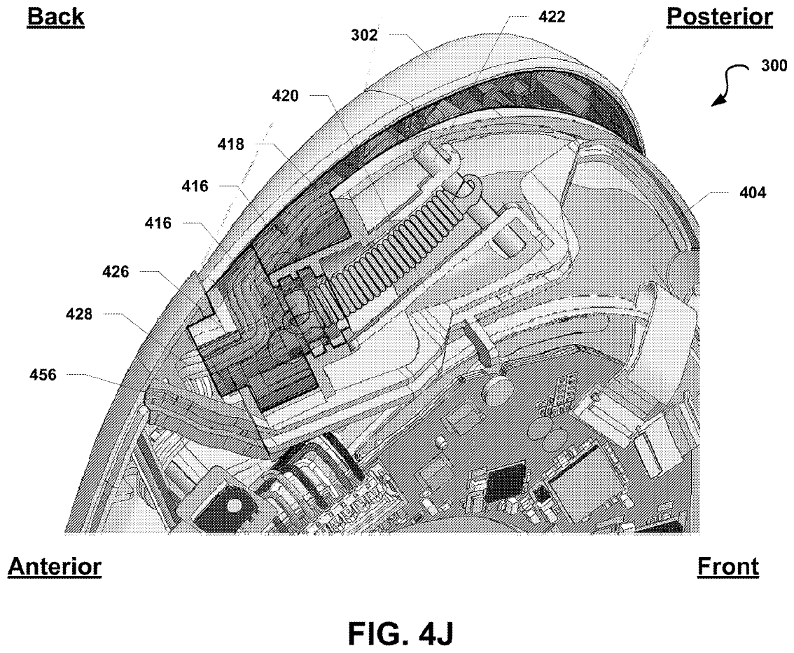

As generally described with reference to the examples illustrated in FIGS. 1A-1H, the ear-worn device 100 may include a plurality of structural features, including without limitation: a hooking body 102 and a device body 104. The hooking body 102 of the ear-worn device 100 may be configured to have a shape that approximates a profile of a root of a posterior portion of a human ear. This shape may be referred to generally as a C-shape. When the hooking body 102 is secured to the user's ear, the hooking body 102 may be positioned proximal to and/or may engage a surface of a root of the posterior and/or top portion of the user's ear. The device body 104 may be configured to have a shape that approximates the profile of a human's outer ear, and as such, the device body 104 may be elliptical or roughly elliptical in shape. In some embodiments, the device body 104 may include or define a mid-ear portion 105 that is substantially positioned at a center or approximate center of the device body 104. In some embodiments, the mid-ear portion 105 may be configured to be suitable for receiving, coupling to, or otherwise accommodating a speaker system that may be mounted to the mid-ear portion 105 (e.g., as further described at least with reference to FIGS. 4A-4J). For example, the mid-ear portion 105 may include one or more fasteners or mounting systems (not shown) that may be configured to couple to corresponding fasteners or mounting systems of a speaker system. In some embodiments, the mid-ear portion 105 may define an opening in the device body 104 that has a shape suitable for receiving or otherwise accommodating at least a portion of a speaker system. While the opening defined by the mid-ear portion 105 is illustrated in various corresponding drawings as having a substantially circular shape, in some embodiments, the mid-ear portion 105 may be configured to define an opening having one of various shapes, including but not limited to, circular, rectangular, elliptical, or various other shapes.

The ear-worn device 100 may include a hinge 106. In some embodiments, the device body 104 may be coupled to the hooking body 102 via the hinge 106. For example, the hinge 106 may be one of various types of hinges (e.g., a tension hinge). The hinge 106 may be configured to couple the device body 104 to the hooking body 102 so that movement of one of the device body 104 or the hooking body 102 is limited in relation to each other. In some embodiments (not shown), the hooking body 102 and the device body 104 may each include complementary magnetic elements that maintain the hooking body 102 and the device body 104 in the closed configured. As such, as the device body 104 is moved towards the hooking body 102, the complementary magnetic elements may pull towards each other, thereby urging the device body 104 towards the hooking body 102.

The hinge 106 may be formed from one or more portions of the hooking body 102 and the device body 104. In some embodiments, the hinge 106 may additionally include one or more other structural features. In a non-limiting example, the hinge 106 may be formed at least in part by a portion of the hooking body 102, a portion of the device body 104, a spring, a first anchor device configured to couple the portion of the hooking body 102 to the spring, and a second anchor device configured to couple the portion of the device body 104 to the spring. In some alternative (or additional) embodiments, the hinge 106 may be a separate structural feature that is separately coupled to the hooking body 102 and the device body 104. In a non-limiting example, the hinge 106 may include a housing configured to couple to a portion of the hooking body 102 and a portion of the device body 104 such that, while the hooking body 102 and the device body 104 are coupled to the hinge 106, the hinge governs the movement of the hooking body 102 and the device body 104 in relation to one another.

With reference to FIGS. 1C-1H, the hinge 106 may be configured to enable the device body 104 to be moved (e.g., swung, rotated, or pivoted) away from the hooking body 102 to cause the ear-worn device 100 to transition from a closed configuration (e.g., as illustrated in FIGS. 1C, 1E, and 1G) to an open configuration (e.g., as illustrated in FIGS. 1D, 1F, and, 1H) by rotating about a rotational axis 108. The hinge 106 may also be configured to enable the device body 104 to be moved (e.g., swung, rotated, or pivoted) back towards the hooking body 102, for example, to transition the ear-worn device 100 from an open configuration to a closed configuration by rotating in the opposite direction along the rotational axis 108.

The hinge 106 may be coupled to the hooking body 102 and the device body 106 so that the rotational axis 106 of the hinge 106 is offset from the hooking body 102 and the device body 106. Rotating the device body 104 about the hinge 106 may cause the device body 104 to change position in three dimensions relative to the hooking body 102. In the examples illustrated in FIGS. 1C-1H, a first dimension is represented by a first directional axis 110, a second dimension is represented by a second directional axis 112, and a third direction is represented by a third directional axis 114.

FIGS. 1C and 1E illustrate different perspectives of the ear-worn device 100 in which the ear-worn device 100 is configured in a closed configuration, according to some embodiments. Specifically, a portion of the device body 104 may be positioned at a first position 122 while the ear-worn device 100 is configured in a closed configuration. By rotating the device body 104 along the rotational axis 108 of the hinge 106, that portion of the device body 104 may change position relative to the hooking body 102 along each of the first directional axis 110, the second directional axis 112, and the third directional axis 114. As such, as illustrated in FIGS. 1D and 1F, the portion of the device body 104 may transition from the first position 122 to a second position 124 by rotating along the rotational axis 108 along a path (e.g., as represented by dotted reference line 126). In these illustrated examples, the first position 122 of the device body 104 may differ from the second position 124 of the device body 104 in three dimensions illustrated by the first, second, and third directional axes 110, 112, 114. For example, as determined in relation to the hooking body 102, a three-dimensional coordinate (e.g., an (x, y, z) coordinate) of the portion of the device body 104 at the first position 122 may differ from a three-dimensional coordinate of the portion of the device body 104 at the second position 124 in each of the three coordinate values. In some embodiments, the position of the device body 104 may only change in two dimensions relative to the hinge 106.

In some embodiments, a degree of change experienced (e.g., a linear distance moved, degrees rotated, and the like) by a portion of the device body 104 when transitioning between a first position and a second position may depend at least in part on a proximity to the hinge 106. When the ear-worn device 100 transitions from a closed configuration to an open configuration, a first portion of the device body 104 farther from the hinge 106 than a second portion of the device body 104 may move to a greater extent in at least one dimension than the extent to which the second portion of the device body 104 moves in the at least one dimension.

In some embodiments, the hinge 106 may be configured to limit the extent to which the device body 104 and the hooking body 102 may be moved toward and away from each other. In the example illustrated in FIG. 1D, the position of the device body 104 relative to the hooking body 102 may depict the furthest extent to which the device body 104 may be moved away from the hooking body 102. Accordingly, in this example, the ear-worn device 100 may be in a “fully open” configuration because the hinge 106 is preventing the device body 104 from being moved further away from the hooking body 102.

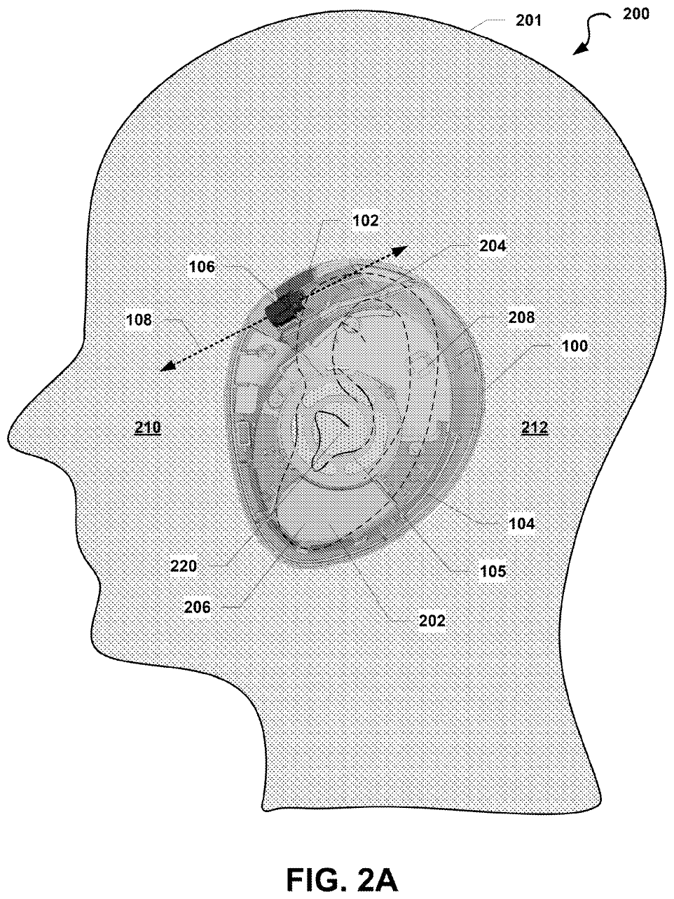

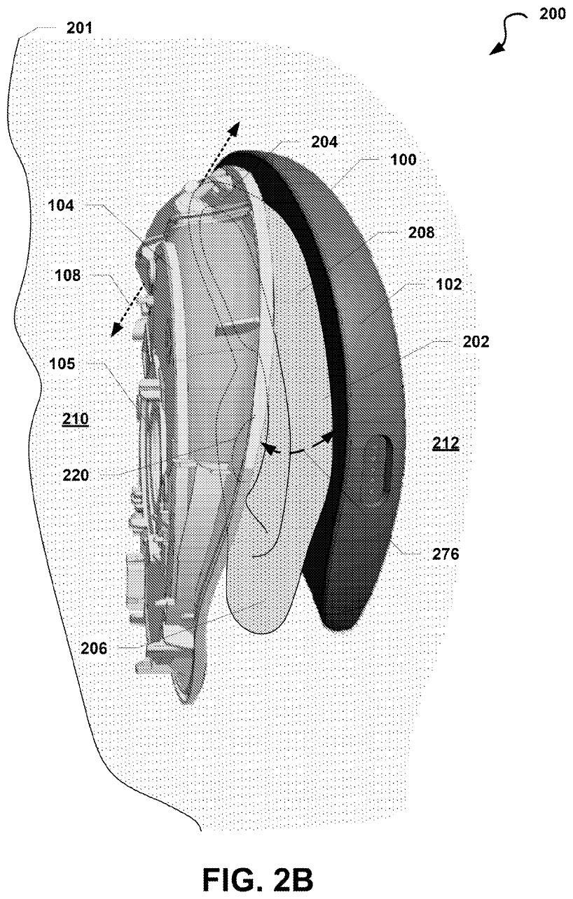

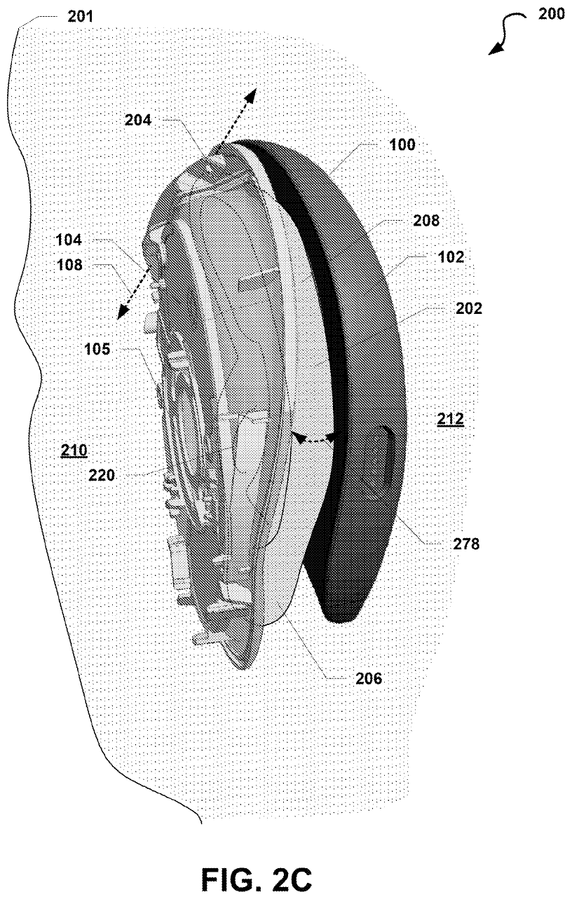

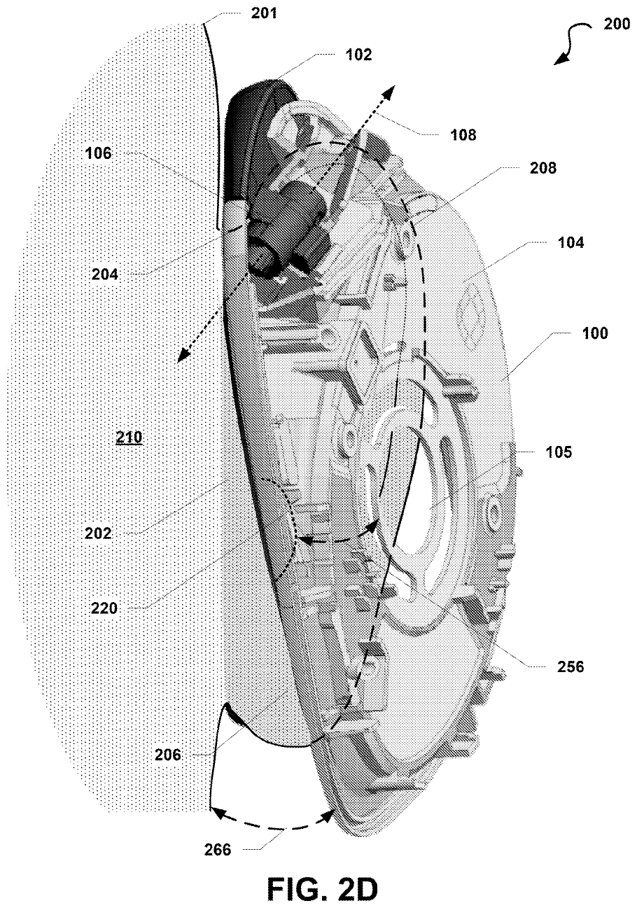

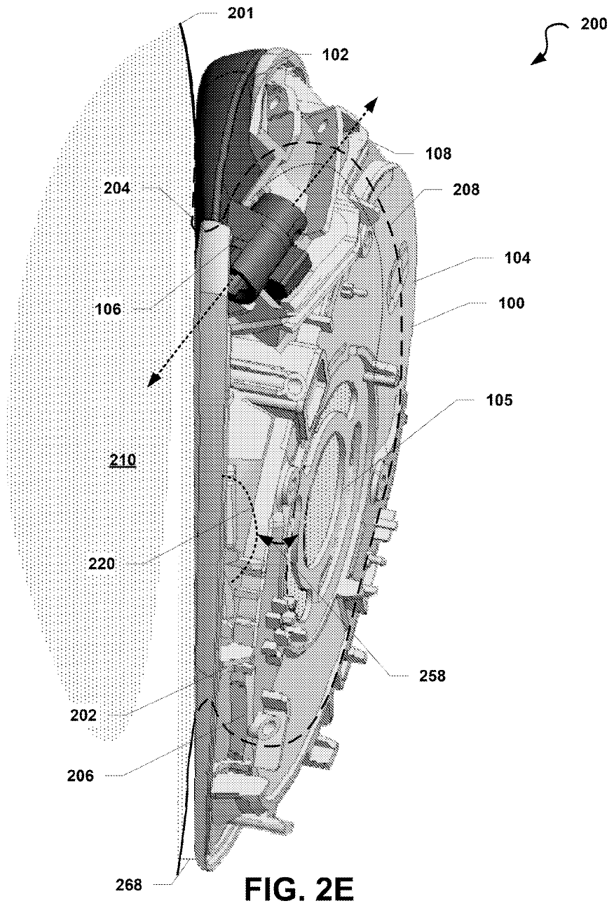

In some embodiments, while the ear-worn device 100 is configured in a closed configuration, a distance between a surface of the device body 104 facing a backward side of the ear-worn device 100 may physically contact or may be near a surface of the hooking body 102 facing a front side of the ear-worn device 102. As the ear-worn device 102 transitions from a closed configuration to an open configuration, a distance between the surface of the device body 104 and the surface of the hooking body 102 may increase so that a space or gap forms between at least a portion of the hooking body 102 and at least a portion of the device body 104. In the example illustrated in FIG. 1G in which the ear-worn device 100 is configured in a closed configuration, a point 142 on a surface of the hooking body 102 may be physically in contact with a point 144 on a surface of the device body 104. However, in the example illustrated in FIG. 1G in which the ear-worn device 100 is configured in an open configuration, the point 142 on a surface of the hooking body 102 may be separated from the point 144 on a surface of the device body 104 (e.g., as indicated by reference line 146). In some embodiments, the gap or space formed between the hooking body 102 and the device body 104 may be suitable for receiving at least a portion of a human ear (e.g., as further described with reference to FIGS. 2A-2E).

In various embodiments described herein, the ear-worn device 100 may be described as transitioning from a closed configuration to an open configuration. However, the ear-worn device 100 may, in some additional or alternative embodiments, may be configured to transition from an open configuration to a closed configuration in a manner opposite of the manner described above with reference to transitioning from a closed configuration to an open configuration.

FIGS. 2A-2E illustrate exterior views of an environment 200 in which an ear-worn device (e.g., the ear-worn device 100 described with reference to FIGS. 1A-1H) is secured to an outer ear 202 of a user 201, according to some embodiments. Specifically, FIG. 2A illustrates an exterior view of a front side of the ear-worn device 100 while the ear-worn device 100 is secured to the outer ear 202 of the user 201, according to some embodiments. FIG. 2B illustrates an exterior perspective view of a posterior side of the ear-worn device 100 while the ear-worn device 100 is worn on the outer ear 202 and configured in an open configuration. FIG. 2C illustrates an exterior perspective view of a posterior side of the ear-worn device 100 while the ear-worn device 100 is secured to the outer ear 202 and configured in a partially closed configuration. FIG. 2D illustrates an exterior view of an anterior side of the ear-worn device 100 while the ear-worn device 100 is worn on the outer ear 202 and configured in an open configuration. FIG. 2E illustrates an exterior view of an anterior side of the ear-worn device 100 while the ear-worn device 100 is secured to the outer ear 202 and configured in a partially closed configuration.

With reference to the example illustrated in FIG. 2A, the ear-worn device 100 may be secured to the outer ear 202, which may include a posterior portion 208, an upper portion 204, an interior portion 220, and a lower portion 206. While the ear-worn device 100 is secured to the outer ear 202, an anterior side of the ear-worn device 100 may face an anterior side 210 of the user 201, and a posterior side of the ear-worn device 100 may face a posterior side 212 of the user 201. In some embodiments, the device body 104 may cover all or substantially all of the user's outer ear 202. Accordingly, when viewed from a front side of the ear-worn device 100, the device body 104 may completely (or substantially) obscure the outer ear 202 when the ear-worn device 100 is secured to the outer ear 202.

In the example illustrated in FIG. 2C, the hinge 106 may urge the device body 104 towards the hooking body 102, and the device body 104 and the hooking body 102 may collectively apply a compressive force to the posterior portion 208 of the outer ear 202 that may ensure that the ear-worn device 100 is secured to the outer ear 202.

The hooking body 102 and the device body 104 of the ear-worn device 100 may be configured collectively so that the ear-worn device 100 may be worn on and secured to the outer ear 202. The ear-worn device 100 may be configured in an open configuration (e.g., by moving the hooking body 102 away from the device body 104 via the hinge 106) so that a space or gap (e.g., a gap 276, FIG. 2B) is present between the hooking body 102 and the device body 104 (e.g., as described with reference to FIGS. 1G-1H). The ear-worn device 100 may then be placed on the outer ear 202 by hooking, hanging, or otherwise positioning the hooking body 102 along the root of the upper portion 204 of the outer ear 202 and by rotating the hooking body 102 until the hooking body 102 engages the root of the posterior portion 208 of the outer ear 202 (obscured by the hooking body 102 in the examples illustrated in FIGS. 2B-2C). Because the ear-worn device 100 features a space or gap between the hooking body 102 and the device body 104 while the ear-worn device 100 is in an open configuration, the posterior portion 208 of the outer ear 202 may move into, at least partially, in such space or gap and remain in such space or gap once the hooking body 102 engages the root of the posterior portion 208 of the outer ear 202 (e.g., as shown in the example illustrated in FIG. 2B). In a non-limiting example (e.g., as illustrated in FIG. 2B), the device body 104 and the rotational axis 108 of the hinge 106 may be configured so that the device body 104 is positioned upward and away from the outer ear 202 while in the ear-worn device 100 is configured in an open configuration. In some further embodiments, padding or other comfortable material may be attached to a surface of the device body 104 that engages the interior portion 220 or another portion of the outer ear 202 to improve comfort while the ear-worn device 100 is secured to the outer ear 202.

While the hooking body 102 is hooked onto the outer ear 202 and while the ear-worn device 100 is configured in an open configuration, the device body 104 may be moved (e.g., swung) along the rotational axis 108 of the hinge towards the hooking body 102. As the device body 104 continues moving towards the hooking body 104, the space or gap between the hooking body 102 and the device body 104 may decrease in at least one dimension (compare, for example, the gap 276 illustrated in FIG. 2B with a gap 278 illustrated in FIG. 2C) until the device body 104 physically contacts at least the posterior portion 208 of the outer ear 202. In some embodiments, once the device body 104 contacts the posterior portion 208 of the outer ear 202, the device body 104 may begin pressing the posterior portion 208 against the hooking body 102, generating a compressive force that secures the posterior portion 208 of the outer ear 202 between the device body 104 and the hooking body 102. For ease of description, the ear-worn device 100 may be described herein as being configured in a partially closed configuration while the posterior portion 208 of the outer ear 202 is secured between the device body 104 and the hooking body 102.

When the device body 104 is moved (e.g., swung) so that the ear-worn device 100 transitions to the closed position, the mid-ear portion 105 of the device body 104 may move into proximity of the interior portion 220 of the outer ear 202. In some embodiments in which a speaker system (not shown) is coupled to the mid-ear portion 105 of the device body 104, the rotational axis 108 of the hinge 106 and the configuration of the device body 104 may cause the speaker system to move nearer to the interior portion 220 of the outer ear 202, thereby enabling the user 201 to experience the speaker system. In the example illustrated in FIG. 2D, the ear-worn device 100 may be configured in an open configuration such that the mid-ear portion 105 of the device body 104 is a first distance (represented by dotted line 256) from the interior portion 220 of the outer ear 202 while the ear-worn device 100 is worn on the outer ear 202. In the example illustrated in FIG. 2E, the ear-worn device 100 may be secured to the outer ear 202 in a partially closed configuration such that the mid-ear portion 105 of the device body 104 is a second distance (represented by dotted line 258) from the interior portion 220 of the outer ear 202. In these examples, the first distance may be greater than the second distance.

Similarly, when the device body 104 is moved so that the ear-worn device 100 transitions to the closed position, a portion of the device body 104 may move towards the anterior side 210 of the user 201. In the example illustrated in FIG. 2D, the ear-worn device 100 may be configured in an open configuration such that a portion of the device body 104 is a third distance (represented by dotted line 266) from the anterior side 210 of the user 201. In the example illustrated in FIG. 2E, the ear-worn device 100 may be configured in a closed configuration in which that portion of the device body 104 is a fourth distance (represented by dotted line 268) from the anterior side 220 of the outer user 201. In these examples, the third distance may be greater than the fourth distance.

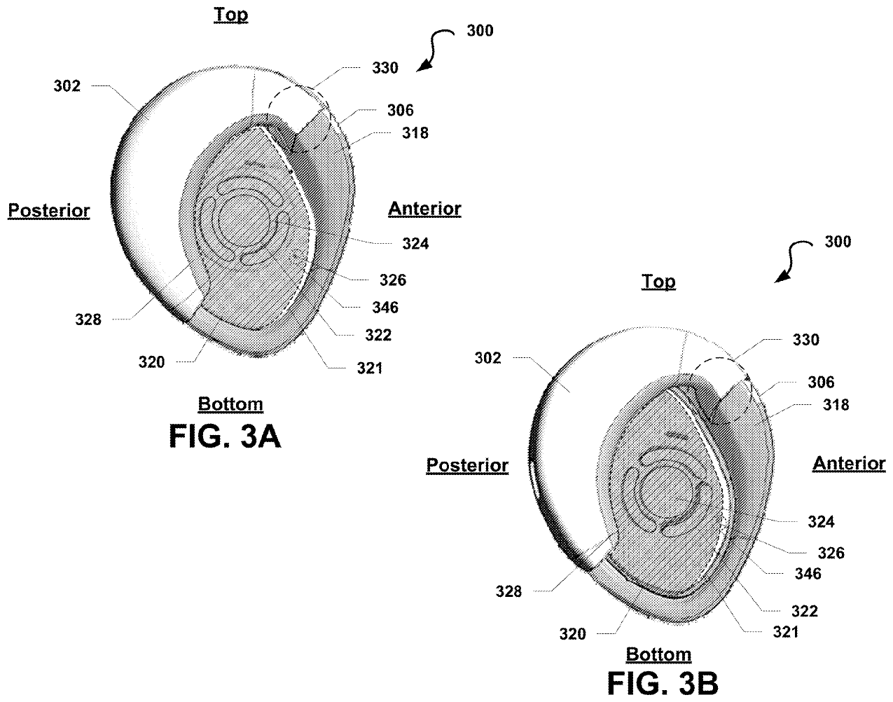

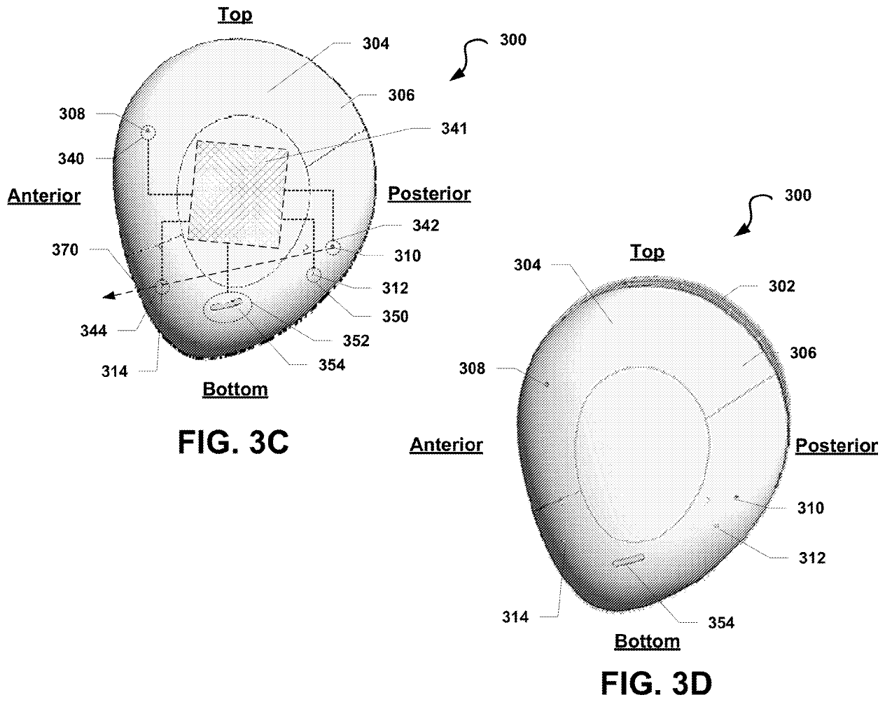

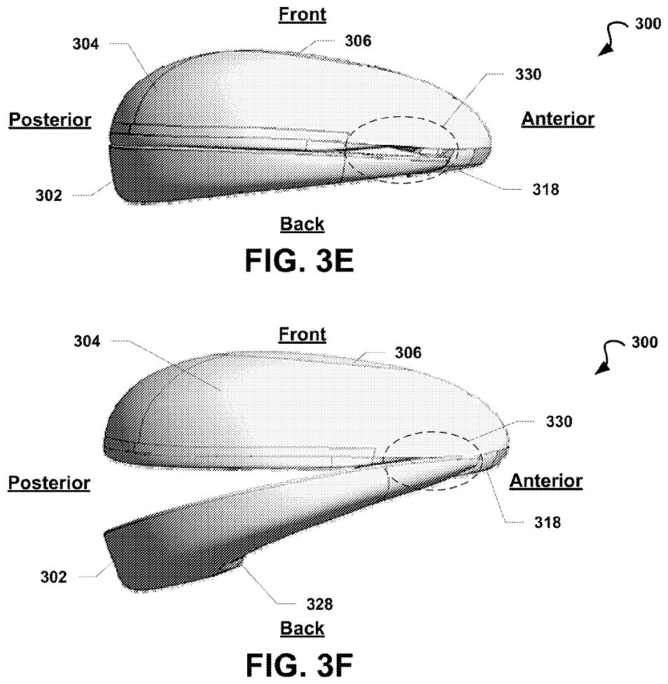

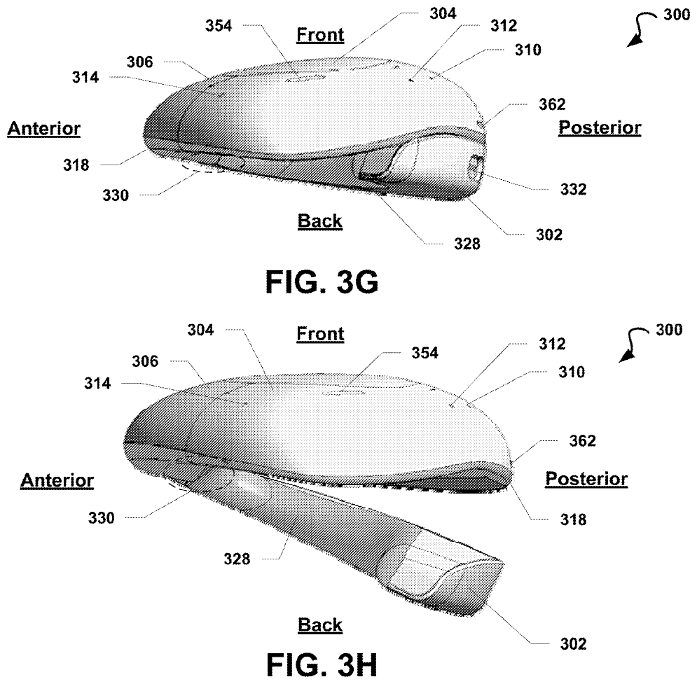

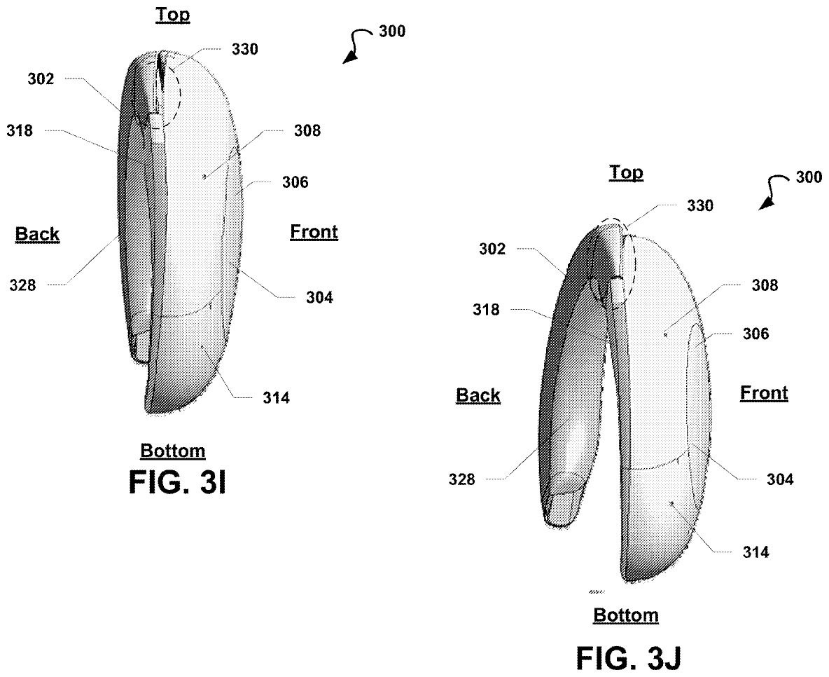

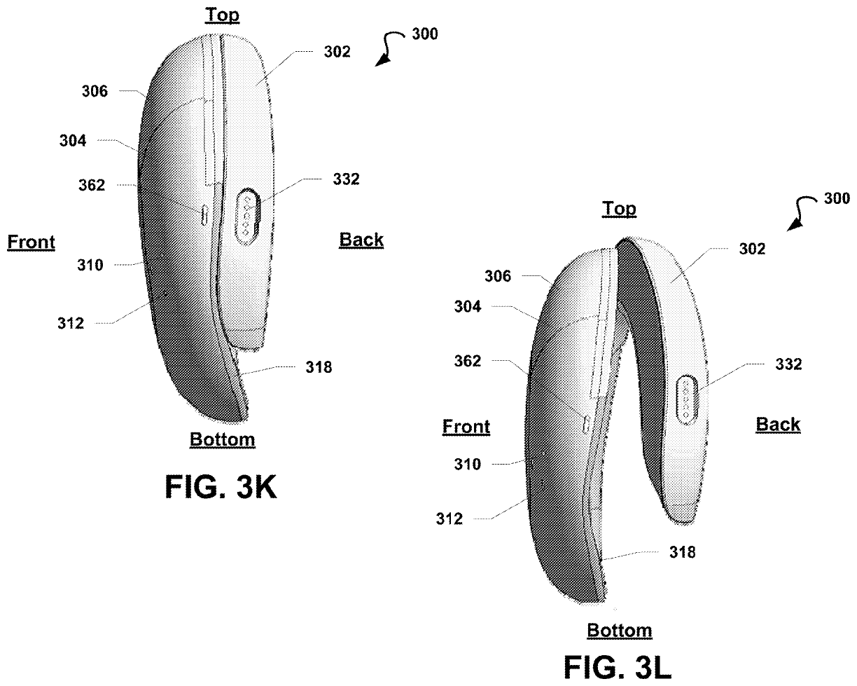

FIGS. 3A-3L illustrate exterior views of an ear-worn device 300 having an alternative design, according to some embodiments. FIG. 3A illustrates an exterior view of a back of the ear-worn device 300 while in a closed configuration. FIG. 3B illustrates an exterior view of a back of the ear-worn device 300 while in an open configuration. FIG. 3C illustrates an exterior view of a front side of the ear-worn device 300 while in a closed configuration. FIG. 3D illustrates an exterior view of a front side of the ear-worn device 300 while in an open configuration. FIG. 3E illustrates a top view the ear-worn device 300 while in a closed configuration. FIG. 3F illustrates a top view the ear-worn device 300 while in an open configuration. FIG. 3G illustrates a bottom view the ear-worn device 300 while in a closed configuration. FIG. 3H illustrates a bottom view the ear-worn device 300 while in an open configuration. FIG. 3I illustrates an anterior side view of the ear-worn device 300 while in a closed configuration. FIG. 3J illustrates an anterior side view of the ear-worn device 300 while in an open configuration. FIG. 3K illustrates a posterior side view of the ear-worn device 300 while in a closed configuration. FIG. 3L illustrates a posterior side view of the ear-worn device 300 while in an open configuration.

With reference to the examples illustrated in FIGS. 3A-3L, the ear-worn device 300 may include a plurality of structural features, including without limitation: a hooking body 302, a hinge 330, and a device body 306. The device body 306 may include a mid-ear portion 322. In some embodiments, the ear-worn device 300 may be configured similarly to the ear-worn device 100 described above (e.g., with reference to FIGS. 1A-2C). Accordingly, in such embodiments, the hooking body 302, the device body 306, the hinge 330, and the mid-ear portion 322 may be configured the same as or similarly to the hooking body 102, the device body 104, the hinge 106, and the mid-ear portion 115 respectively described above (e.g., with reference to FIGS. 1A-2E). For ease of description, duplicative descriptions of such configurations of the hooking body 302, the device body 306, the hinge 330, and/or the mid-ear portion 322 may be omitted from the following descriptions.

In some embodiments, the hooking body 302 may be coupled to the device body 306 via the hinge 330. The hinge 330 may be configured to enable the device body 306 to be moved (e.g., swung, rotated, or pivoted) away from the hooking body 302 to cause the ear-worn device 300, for example, to transition from a closed configuration (e.g., as illustrated at least in FIG. 3A) to an open configuration (e.g., as illustrated at least in FIG. 3B) by rotating about a rotational axis (not shown). The hinge 330 may also be configured to enable the device body 306 to be moved (e.g., swung, rotated, or pivoted) back towards the hooking body 302, for example, to transition the ear-worn device 300 from an open configuration to a closed configuration by rotating in the opposite direction along the rotational axis.

In some embodiments, the hinge 330 may urge the hooking body 302 and the device body 306 together. For example, the hinge 330 may include a torsion spring or another biasing member that causes the hooking body 302 and the device body 306 to move towards each other in the absence of an external force. The hinge 330 may be configured to couple the device body 306 to the hooking body 302 so that movement of one of the device body 306 or the hooking body 302 is limited in relation to each other. In some optional embodiments, the hooking body 302 and the device body 306 may each include complementary magnetic elements that maintain the hooking body 302 and the device body 306 in a closed configuration. As such, as the device body 306 is moved towards the hooking body 302, the complementary magnetic elements may pull towards each other, thereby urging the device body 306 towards the hooking body 302.

In some embodiments (not shown), while in the open configuration, the ear-worn device 300 may be suitable for receiving at least a posterior portion of a user's ear in a space formed between the hooking body 302 and the device body 306 (e.g., as similarly described with reference to FIGS. 2A-2E). The hinge 330 may then cause the ear-worn device 300 to transition to a closed configuration that may secure the ear-worn device 300 to the user's ear by securing at least the posterior portion of the user's ear between the hooking body 302 and the device body 306 while the hooking body 302 rests on and/or is secured to an upper root portion of the user's ear. In some embodiments (not shown), while in the closed configuration, the mid-ear portion 322 may be near the interior portion of the user's outer ear.

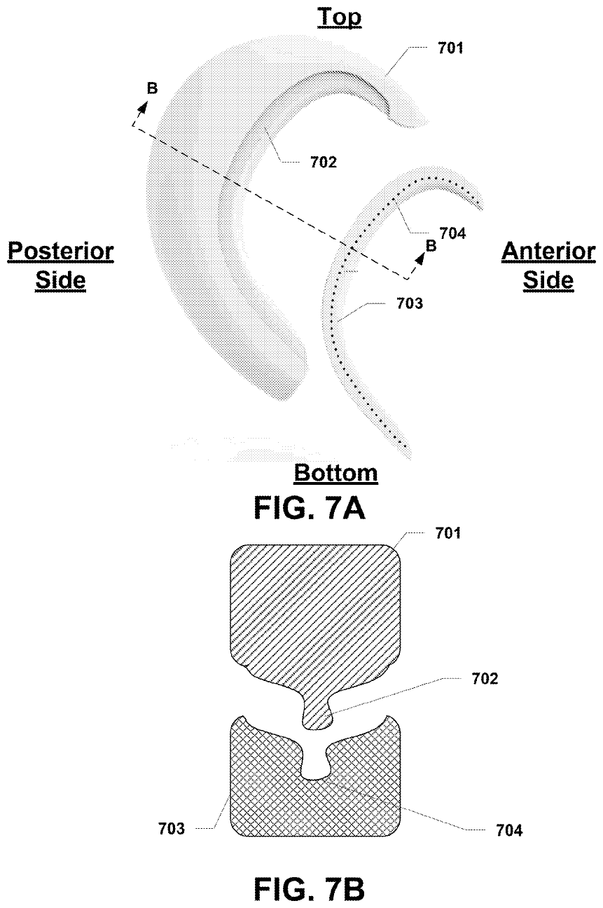

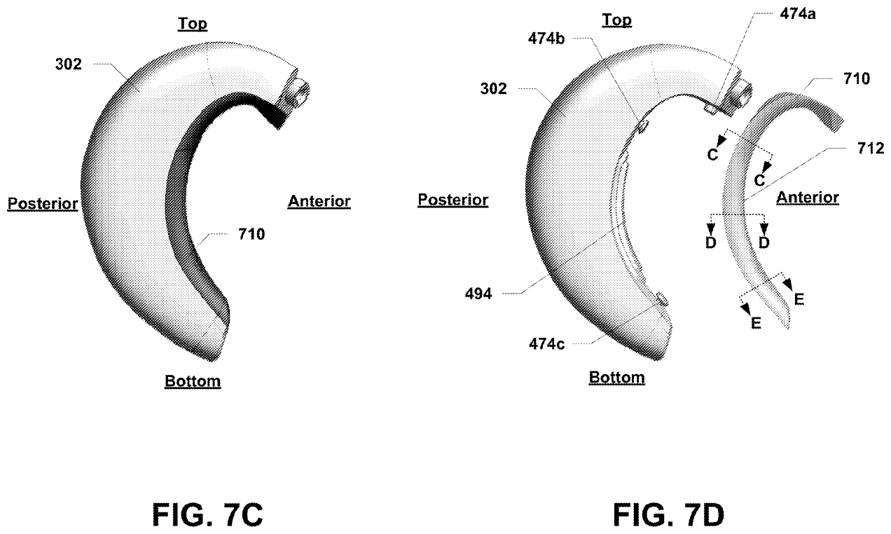

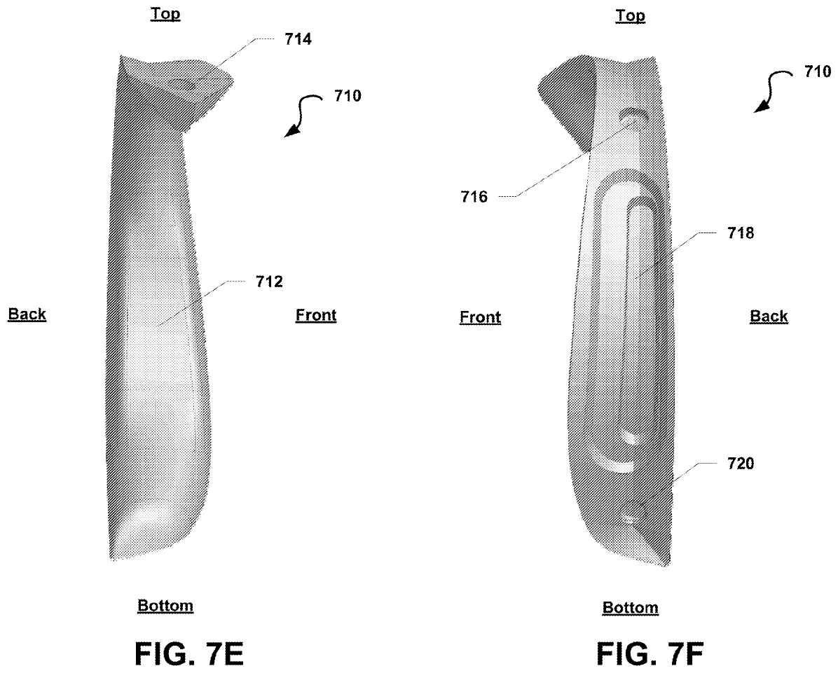

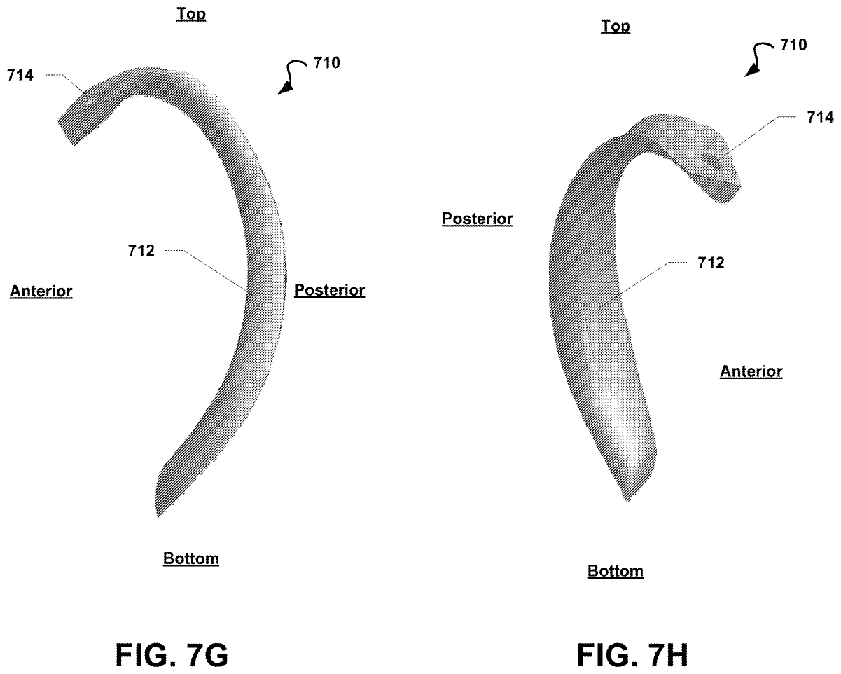

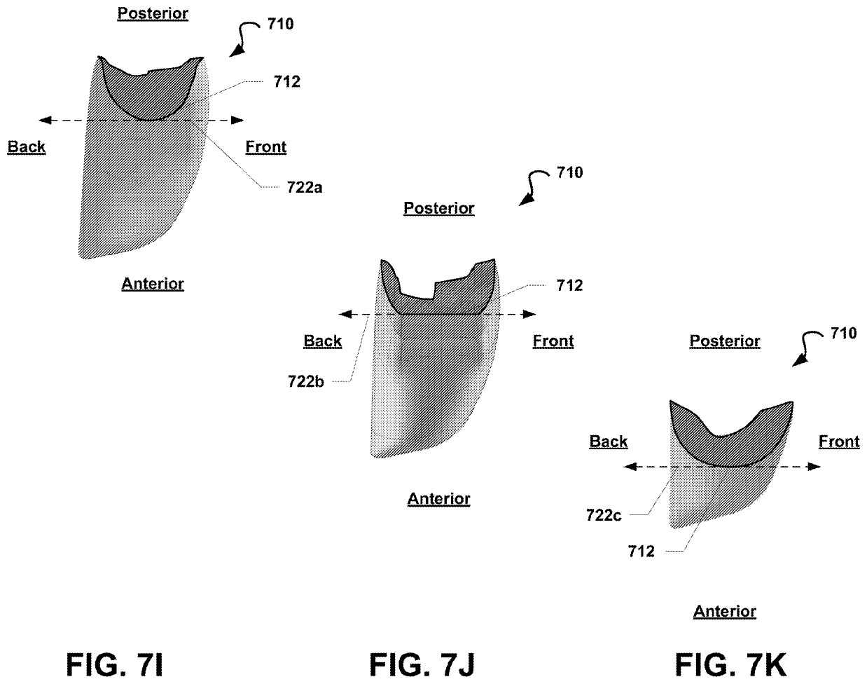

In some embodiments, the hooking body 302 may be configured to be couplable to one or more ear-fitting attachments (e.g., an ear-fitting attachment 328 illustrated in at least FIG. 3A) via one or more coupling devices (e.g., fasteners) and/or coupling techniques. In some embodiments, the ear-fitting attachment 328 may be couplable to a portion of the hooking body 302 facing an anterior side of the ear-worn device 300. In some embodiments, the ear-fitting attachment 328 may be removed/decoupled from the hooking body 302 by unfastening or overcoming a coupling force causing the ear-fitting attachment 328 to remain coupled to the hooking body 302. In some embodiments, the ear-fitting attachment 328 may be configured to have a shape suitable for engaging a surface of a root of a posterior portion of a user's ear (e.g., a “C” shape). Additional (or alternative) embodiments of the hooking body 302 and the ear-fitting attachment 328 are described herein (e.g., with reference to FIGS. 7A-7K).

The device body 306 may be coupled to or may include an ear pad (e.g., a partially transparent ear pad 320 as illustrated in at least FIG. 3A). In some embodiments, the ear pad 320 may be coupled to, attached to, or positioned towards a back-facing side of the device body 306 (e.g., as illustrated in at least FIG. 3A). The ear pad 320 may include or may be made from one or more materials, such as one or more soft, pliable materials suitable for physically engaging a human ear. In some embodiments, while the ear-worn device 300 is configured in an open configuration, a posterior portion of the user's ear may be inserted between the hooking body 302 and the device body 306 (e.g., as described above). When the ear-worn device 300 transitions from an open configuration to a closed configuration, the device body 306 may move towards the hooking body 302, thereby causing the ear pad 320 to physically engage the user's ear. In some embodiments, the pliability of the ear pad 320 may cause the ear pad 320 to conform to the anatomical features of the auricle surrounding the interior portion of the user's ear (e.g., an area near the user's ear canal) and, as a result, may form at least a partial acoustic seal that mitigates or prevents ambient sound from reaching the user's ear canal while the ear-worn device 300 is secured to the user's ear. By way of a non-limiting example, the ear pad 320 may press against and conform to at least the tragus, antihelix, antitragus, and other surrounding anatomical features of the user's outer ear.

In some embodiments, the device body 306 may include or be coupled to a first speaker system 324. The first speaker system 324 may be obscured by (e.g., covered by) the ear pad 320. In some embodiments, the first speaker system 324 may be configured to produce sound that is directed through the ear pad 320. In such embodiments, the ear pad 320 may include or may be made from one or more acoustically transparent materials, such as acoustically transparent foam. An acoustically transparent material is a material that enables sound (or certain frequencies) of sound to pass without attenuating the sound or by only slightly attenuating the sound. Thus, in such embodiments, the first speaker system 324 may produce sounds towards the ear pad 320, and the sound may pass without attenuation (or only slightly attenuated) towards the ear canal of the user's ear.

In some embodiments, the device body 306 may include or be coupled to an edge member 318. The edge member 318 may include or be made from one or more materials that are suitable for physically engaging a user's ear and/or portions of the user's face. In such embodiments, while the ear-worn device 300 is secured to a user's ear, the edge member 318 may press against the user's ear and/or portions of the user. In some embodiments, the edge member 318 may be configured to form a slight recessed area or space (e.g., as illustrated in FIG. 3A), and the ear pad 320 may be positioned within this recessed area or space. In some additional or alternative embodiments, the ear pad 320 may be configured so that the ear pad 320 fills the recessed area or space and does not extend beyond the edge member 318 towards the hooking body 302.

In some embodiments the ear pad 320 may be selectively coupled to the device body 306. For example, the ear pad 320 may be coupled to the device body 306 and may be decoupled from the device body 306 (e.g., to facilitate cleaning or replacement of the ear pad 320). In some embodiments, the ear pad 320 may be held in place within the slight recessed area or space formed by the edge member via a ring member 321. In such embodiments, the ring member 321 may be configured to have a shape that is the same or substantially similar to an edge or perimeter of the slight recessed area or space formed by the edge member 318, and the ring member 321 may be configured to be inserted between an edge of the edge member 318 that forms the recessed area or space and the ear pad 320. Once inserted, the ring member 321 may hold the ear pad 320 in place within the recessed area or space formed by the edge member 318. In some embodiments, the ring member 321 may secure the ear pad 320 to the device body 306 via friction, fasteners, or the like.

In some embodiments (e.g., as illustrated in at least FIGS. 3C-3D), the device body 306 may include a device cover 304. The device cover 304 may be made from one or more materials or a combination of one or more materials, such as one or more types of plastic. In some embodiments, the device body 306 may include a touch-sensitive sensor or sensors (not shown) under the device cover 304. By way of a non-limiting example, the touch sensitive sensor or sensors may be a capacitive touch sensor or one or more other touch sensitive sensors known in the art. In such embodiments, the device cover 304 may be made from a material suitable for enable the touch-sensitive sensor or sensors to measure changes in electrical properties, such as when a user's finger touches the device cover 304.

In some embodiments (e.g., as illustrated in FIG. 3C), the device body 306 may include one or more electronic components, such as a processing unit 341, a first microphone 346 (e.g., as depicted in the example illustrated in FIG. 3A), a second microphone 340, a third microphone 342, a fourth microphone 344, a lighting element 350, and a second speaker system 352. The processing unit 341 may also be in electrical communication with the microphones 346, 340, 342, 344, the lighting element 350, the first speaker system 324 (e.g., as depicted in FIG. 3A), and the second speaker system 352. The processing unit 341 may receive input from one or more of the above electrical components and may send signals to one or more of the above electrical components to control, change, activate, or deactivate operations of one or more of the above electrical components. In some embodiments, the processing unit 341 may include or a digital signal processor or another processor that may be configured to receive and process audio signal inputs from one or more of the microphones 346, 340, 342, 344. The processing unit 341 may also be configured to provide audio signals to one or both of the speaker systems 324, 352 to cause those speaker systems 324, 352 to output the audio signals as sound.

In some embodiments, the first microphone 346 may be included or embedded in the device body 306 near the first speaker system 324 and may be configured to capture sound from the first speaker system 324 via a first opening 326. The first microphone 346 may provide audio signals of the sound captured from the first speaker system 324 to the processing unit 341. The processing unit 341 may utilize those audio signals to perform one or more known active-noise-cancelling techniques. In some embodiments, the first microphone 346 may be positioned underneath or may be obscured by the ear pad 320 (e.g., as illustrated in FIG. 3A). In some additional or alternative embodiments, the processing unit 341 may utilize audio signals generated by one or more of the other microphones 340, 342, 344 to perform active noise cancellation. For example, the processing unit 341 may receive audio signals representative of ambient sound from one or more of the microphones 340, 342, 344 and may utilize these audio signals to modify audio signals provided to one or both of the speaker systems 324, 352, for example, to cancel the ambient noise by generating 180-degrees-out-of-phase anti-noise signals as would be known by one skilled in the arts.

In some embodiments, the device cover 304 may be configured to include a second opening 308, a third opening 310, and a fourth opening 314. Each of the openings 308, 310, 314 may be formed as one or more openings in the device cover 304 that may permit ambient sound to pass through the openings and to be captured by the second, third, and fourth microphones 340, 342, 344, respectively. In some embodiments, at least two of the microphones 340, 342, 344 and their respective openings 308, 310, 314 may be positioned along an axis so that the processing unit 341 may utilize audio signals generated from those at least two microphones to perform beamforming and/or noise-cancellation techniques. For example (e.g., as illustrated in FIG. 3C), the microphones 344 and 342 may be positioned along an axis (e.g., as represented by the dotted, arrow line 370) that extends towards a user's mouth while the user is wearing the ear-worn device. In this example, the processing unit 341 may receive audio signals from at least these two microphones 344 and 342 and may perform beamforming/noise-cancellation to improve the quality of a user's voice that is captured by those microphones. In some embodiments, the device cover 304 may include an opening 354 in the device cover 304. The opening 354 may include one or more opening that are suitable for enabling sound generated from the second speaker system 352 to pass through the opening 354 into the surroundings.

The lighting element 350 may be one of various types of lighting devices, such as a light-emitting diode. In some embodiments, the processing unit 341 may control various characteristics of the lighting element 350, including activating/deactivating the lighting element 350, causing the lighting element 350 to display one or more colors or combinations of colors, and the like.

FIGS. 4A-4J illustrate partial interior views of the ear-worn device 300 described above (e.g., with reference to FIGS. 3A-3L), according to some embodiments. FIG. 4A illustrates a front view of the ear-worn device 300 in which the device cover 304 is decoupled to illustrate an arrangement of components included in or coupled to the device body 306, according to some embodiments. FIG. 4B illustrates a bottom view of the ear-worn device 300 in which the device cover 304 is decoupled from the device body 306, according to some embodiments. FIG. 4C illustrates a posterior view of the ear-worn device 300 in which the device cover 304 is decoupled from the device body 306, according to some embodiments. FIG. 4D illustrates a top view of the ear-worn device 300 in which the device cover 304 is decoupled from the device body 306, according to some embodiments. FIG. 4E illustrates a back view of the ear-worn device 300 in which the hooking body 302 is partially transparent to show an interior of the hooking body 302, according to some embodiments. FIG. 4F illustrates a back-perspective view of the ear-worn device 300 in which the ear-worn device 300 is configured in a closed configuration, the hooking body 302 is partially transparent, and the device cover 304 is decoupled from the device body 306, according to some embodiments. FIG. 4G illustrates another back-perspective view of the ear-worn device 300 in which the ear-worn device 300 is configured in an open configuration, the hooking body 302 is partially transparent, and the device cover 304 is decoupled from the device body 306, according to some embodiments. FIG. 4H illustrates a top perspective view of the ear-worn device 300 in which the ear-worn device 300 is configured in a closed configuration, the hooking body 302 is partially transparent, and the device cover 304 is decoupled from the device body 306, according to some embodiments. FIG. 4I illustrates another back-perspective view of the ear-worn device 300 in which the ear-worn device 300 is configured in an open configuration, the hooking body 302 is partially transparent, and the device cover 304 is decoupled from the device body 306, according to some embodiments. FIG. 4J illustrates a close-up view of portions of the hinge 330, device body 306, and hooking body 302 of the ear-worn device 300 in which the ear-worn device 300 is configured in an open configuration.

With reference to FIGS. 4A-4J, the device body 306 may include a device body substrate 404. The device body substrate 404 may be made from one or more materials, or a combination of materials, that may be suitable for be coupled to one or more components (e.g., as further described herein). By way of a non-limiting example, the device body substrate 404 may be made from or may include one or more of plastic, metal, or hard rubber. In some embodiments, the device body 306 may be coupled to or may include any combination (including an empty set)—but is not limited to including or being coupled to—of the following: a flexible circuit board 402, a printed circuit board 406, a central processing unit 403, a touch sensor 408, a digital signal processor 410, a coupling channel 412, a joint socket 416, a spring 418, a spring channel 420, a first spring anchor 422, a second spring anchor 424, electrical leads 428, an electrical connector 430, a speaker housing 432, a near-filed magnetic induction (NFMI) coil 438, a magnetic sensor 442, a magnetic element 444, at least one radiofrequency (RF) transceiver 450, a flexible circuit 452, a flexible circuit 454, a flexible circuit 458, and an acoustic barrier 456.

In some embodiments, the printed circuit board 406 may be coupled to the device body substrate 404. The printed circuit board 406 may be coupled to the central processing unit 403, the digital signal processor 410, and the electrical connector 430. In some embodiments, the printed circuit board 406 may receive power and/or other signals from the electrical connector 430. The electrical connector 430 may be electrically coupled to a power source (e.g., a battery as illustrated in at least FIG. 4E) via one or more of the electrical leads 428. The printed circuit board 406 may be configured to distribute power from the electrical connector 430 to one or more components coupled directly or indirectly to the printed circuit board 406 as would be known by one skilled in the art.

In some embodiments, the printed circuit board 406 may be coupled to the flexible circuit 402. The flexible circuit 402 may be coupled to the second microphone 340, the third microphone 342, the fourth microphone 344, and the lighting element 350 (e.g., as described above with reference to one or more of FIGS. 3A-3L). The printed circuit board 406 may additionally (or alternatively) be coupled to the flexible circuit 452. The flexible circuit 452 may be coupled to the touch sensor 408, which may be configured to receive touch inputs from a user. For example, the touch sensor 408 may be configured as a capacitance touch sensor and may detect touch input received on the device cover 406 (not shown). In some embodiments, the printed circuit board 406 may be coupled to the flexible circuit 458, which may be coupled to the first speaker system 324.

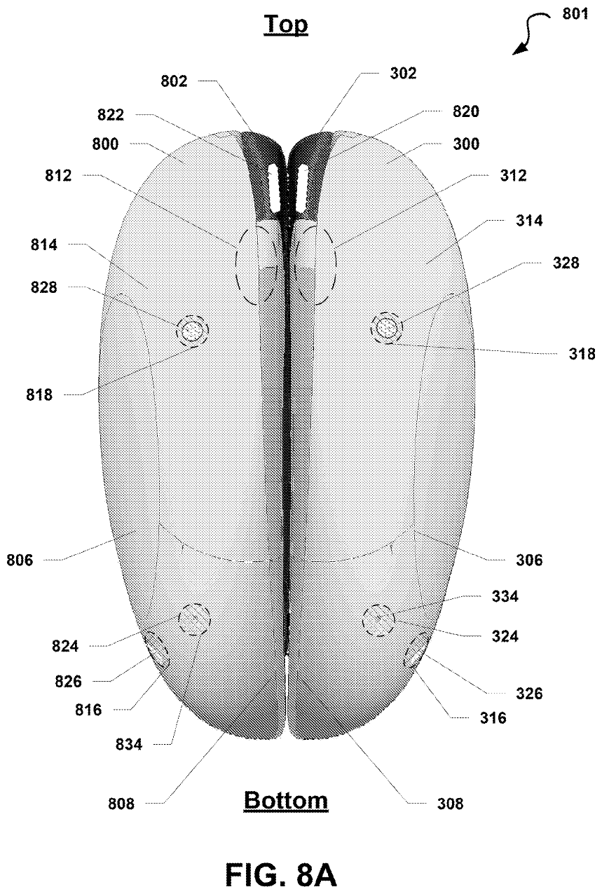

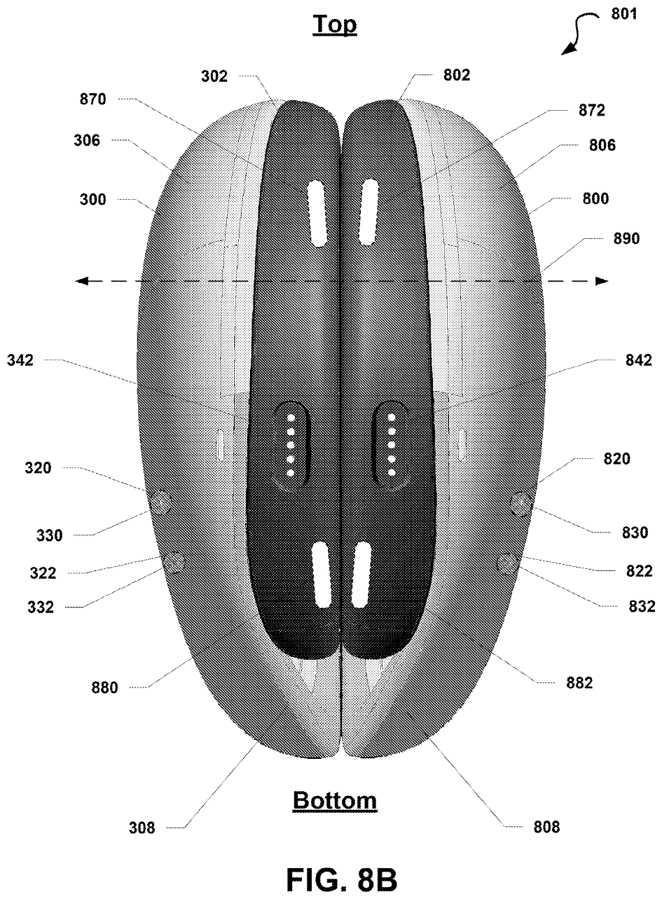

As described (e.g., above), the ear-worn device 300 may be used in conjunction with another ear-worn device 300. In some embodiments, the NFMI coil 438 may be configured to transmit and receive signals and other information from a corresponding NFMI coil in another ear-worn device (not shown). In some embodiments, the magnetic sensor 440 may be configured to detect when a magnetic element (e.g., a permanent magnet) included in the other ear-worn device 300 is placed near the magnetic sensor, such as when the ear-worn device 300 and the other ear-worn device are placed side by side (e.g., as illustrated in FIGS. 8A-8B). Similarly, a magnetic sensor included on the other ear-worn device 300 may be configured to detect when the magnetic element 444 is near that magnetic sensor.

In some embodiments (e.g., as illustrated with reference to FIGS. 4E-4I), the hooking body 302 may be coupled to or may include a hooking body substrate 492. The hooking body substrate 492 may be made from one or more materials, or a combination of materials, that may be suitable for be coupled to one or more components (e.g., as further described herein). By way of a non-limiting example, the hooking body substrate 492 may be made from or may include one or more of plastic, metal, or hard rubber. In some embodiments, the hooking body 302 (or specifically the hooking body substrate 492) may be coupled to or may include any combination (including an empty set)—but is not limited to including or being coupled to—of the following: a first device coupling element 484, a second device coupling element 486, a first charger coupling element 480, a second charger coupling element 482, a power charging connector 476, a battery 478, and a joint pin 426.

In some embodiments, the device coupling elements 484, 486 may be configured to couple the hooking body 302 to another ear-worn device (e.g., as illustrated in FIGS. 8A-8B). The device coupling elements 484, 486 may be or may include one or more fasteners or coupling devices. By way of a non-limiting example (and as further described with reference to FIGS. 8A-8B), the device coupling elements 484, 486 may be magnetic elements—e.g., permanent magnets or ferrous materials) that may be magnetically coupled to complementary device coupling elements included on or in a hooking body of another ear-worn device.

In some embodiments, the battery 478 may be embedded within the hooking body 302. As illustrated (e.g., with reference to FIG. 4E), the battery 478 may be configured to have a shape that at least partially conforms to a shape of the hooking body 478, and as such, the battery 478 may have at least surface that has a partially curved shaped. In some embodiments, at least one electrical lead 428 may be electrically connected to the battery 478 in the hooking body 302. The at least one electrical lead 428 may pass through the hinge 330 into the device body 306. In some embodiments (e.g., as described above), the at least one electrical lead 428 may provide power from the battery 478 to various electrical components included in the device body 306, such as the first speaker system 324 included in or attached to the device body 306. In some embodiments, the battery 478 may protrude towards the anterior side of the hooking body 302 into a protruding portion of the hooking body 494.

The power charging connector 476 may be configured to receive power from an external power supply. In a non-limiting example, the power charging connector 476 may mate with a reciprocal charging connector include as part of a charging cable (not shown). The power charging connector 476 may receive power from the reciprocal charging connector and may route the power to the battery 478 or to one or more other electrical components included in the hooking body 302 and/or the device body 306. In some embodiments, the charger coupling devices 480, 482 may be configured to couple the hooking body 302 to an external charging connector that is compatible with the power charging connector 476. In some additional (or alternative) embodiments, the charger coupling devices 480, 482 may be or may include magnetic elements that magnetically attach to other magnetic elements included in the external charger.

In some embodiments, the hooking body 302 may include one or more coupling members. In the example illustrated in FIG. 4E, the hooking body 302 includes a first coupling member 474a, a second coupling member 474b, and a third coupling member 474c. In some embodiments, the coupling members 474a-474c may be configured to enable the hooking body to couple to the sizing attachment 328 (e.g., illustrated as partially transparent in FIG. 4E). Specifically, each of the coupling members 474a-474c may couple to a complementary coupling member or device included in the sizing attachment 328 (not shown). In the example illustrated in FIG. 4E, the coupling members 474a-474c are configured as male coupling devices that may couple to female coupling devices included in the sizing attachment 328 (e.g., via friction, buttons, fasteners, magnets, or the like). In some embodiments, the coupling members 474a-474c may be uncoupled from the sizing attachment 328 to facilitate cleaning or replacement.

Returning to the examples illustrated in FIGS. 4A-4J, a spring 418 may be coupled to the device body 306 via the first spring anchor 422. For example, the spring 418 may be configured to include an eye loop at one end of the spring 418, and the first spring anchor 422 may couple the spring 418 to the device body 306 by inserting the first spring anchor 422 through a first opening of the spring channel 420, through the eye loop of the spring 418, and through a second opening of the spring channel 420. In some embodiments, the spring channel 420 may be configured to receive the body of the spring 420.

In some embodiments, the hooking body 302 may include a joint pin 426, which may be coupled to or formed as part of an end of the hooking body 302. The joint pin 426 may be configured to have a tubular shape. The joint pin 426 may be coupled to the device body 306 via the joint socket 416, which may, in some embodiments, be formed as part of the device body 306. In some additional (or alternative) embodiments, the joint pin 426 may include a portion that is configured to rest within the joint socket 416 and may include at least one opening suitable for receiving the second spring anchor 424. By way of a non-limiting example, the joint pin 426 may include one or more flanges that may be fitted within one or more corresponding grooves in the joint socket 416. In some alternative embodiments, the joint pin 426 may not include one or more flanges and may instead be configured to have a substantially cylindrical shape. Specifically, while the spring 418 is coupled to the spring channel 420 via the first spring anchor 422 and positioned within the spring channel 420, an eye loop at a second end of the spring 418 may be coupled to the joint pin 426 by inserting the second spring anchor 424 through an opening in the joint pin 426, through the eye loop at the second end of the spring 418, and through another opening in the joint pin 426. In some embodiments, while the spring 418 is coupled to the spring channel 420 via the first spring anchor 422, a desired amount of torque may be loaded on the spring 418 prior to coupling the spring 418 to the hooking body 302 to ensure that the spring 418 acts on the hooking body 302 and/or the device body 306 with the desired amount of torque. In some embodiments, because the spring 418 may be substantially cylindrical and the spring channel 420 may be at least partially curved, the spring 418 may be deformed into the at least partially curved shape of the spring channel 420 when coupled to the hooking body 302 and the device body 306 via the spring anchors 422 and 424.

In some embodiments, while the ear-worn device 300 is configured in a closed configuration, the spring 418 may be configured in a low- or no-tension state. When the ear-worn device 300 transitions from a closed configuration to the open configuration as a result of an applied external force, the joint pin 426 may rotate about an axis (e.g., as described with reference to FIGS. 1A-2E). Because the spring is coupled to the joint pin 426, an end of the spring 418 coupled to the joint pin 426 may begin to rotate about the same axis. However, because the other end of the spring 418 is coupled to the device body 306, such end of the spring 418 may be unable to rotate (or may have its rotation impeded), thereby producing a tension within the spring 418. Accordingly, when the applied external force is removed from the hooking body 302, the tension of the spring 418 may cause the end of the spring 418 coupled to the joint pin 426 to rotate in the opposite direction, which may cause the joint pin 426 and the hooking body 302 to also rotate in that opposite direction until the ear-worn device 300 returns to a closed configuration.

In some embodiments, the coupling channel 412 may be configured as a grooved feature that follows at least a portion of the device body 306 (e.g., as illustrated in at least FIG. 4A). In such embodiments, the coupling channel 412 may be configured to couple with a portion of the device cover 304 such that the portion of the device cover 304 may be inserted into the groove formed by the coupling channel 412. In some embodiments, an adhesive substance may be applied to the coupling channel 412 prior to coupling with the portion of the device cover 304 to permanently couple the device cover 304 to the device body 306. In such embodiments, when the device cover 304 is coupled to the device body 306, the coupling channel 412 and the device cover 304 may form, at least substantially, an at least substantially closed volume of air around the first speaker system 324. This at least substantially closed volume of air may function as a back volume for the first speaker system 324, which may improve the quality of sound generated by the first speaker system 324.

In some embodiments, the second speaker system 352 may be included in the speaker housing 432. In some embodiments, the speaker housing 432 may be coupled to or formed as part of the device body 306 (or, specifically, the device body substrate 404) and may at least substantially encapsulate the second speaker system 352. In such embodiments, the speaker housing 432 may include a first volume of air that is at least substantially isolated from the second volume of air formed when the device cover 304 is coupled to the coupling channel 412 (e.g., as described above). Because the first volume of air is at least substantially isolated from the second volume of air, the sound generated via the second speaker system 352 may not affect (or may only slight affect) sound generated via the first speaker system 324, and vice versa. In some embodiments, the speaker housing 432 may be configured to have a shape such that sound generated via the second speaker system 352 has one or more desired acoustic characteristics.

As described, the one or more electrical leads 428 may run from the hooking body 302 (e.g., from the battery 478 in the hooking body 302) to the electrical connector 430 included in the device body 306. In some embodiments, the one or more electrical leads 428 may pass through the hinge 330. By way of a non-limiting example, the joint pin 426 may include a hollow center, and the one or more electrical leads 428 may pass through the hollow center of the joint pin 426. By running through the joint pin 426, the one or more electrical leads 428 may not experience a significant amount of rotational stress (e.g., shearing stress) within the joint pin 426 even while the joint pin 426 is rotating. By way of a non-limiting example, the one or more electrical leads 428 may experience 15 or fewer degrees of rotational stress when the ear-worn body 300 transitions from a closed configuration to an open configuration. Because the rotational stress is relatively small, the one or more electrical leads 428 may have increased longevity before wearing out.

In some additional (or alternative) embodiments, the one or more electrical leads 428 may pass through the hollow center of the joint pin 426 and pass through an opening in the acoustic barrier 456, which may be positioned within a channel formed in the device body substrate 404. When the device cover 304 is coupled to the device body 306, the acoustic barrier 456 may be configured to, in conjunction with the device body substrate 404 and the device cover 304, created a sealed volume of air. As such, the one or more electrical leads 428 may enter the volume of air without compromise the acoustical seal formed around the first speaker system 324 by the acoustic barrier 456, device cover 304, and device body substrate 404 (e.g., as described above).

The device body substrate 404 may be coupled to a speaker housing 432. In some embodiments, the speaker housing 432 may encapsulate the second speaker 352 (e.g., as described with reference to FIGS. 3A-3L). The speaker housing 432 may be formed as an at least partially sealed acoustic chamber configured to function as a back volume for the second speaker 352.

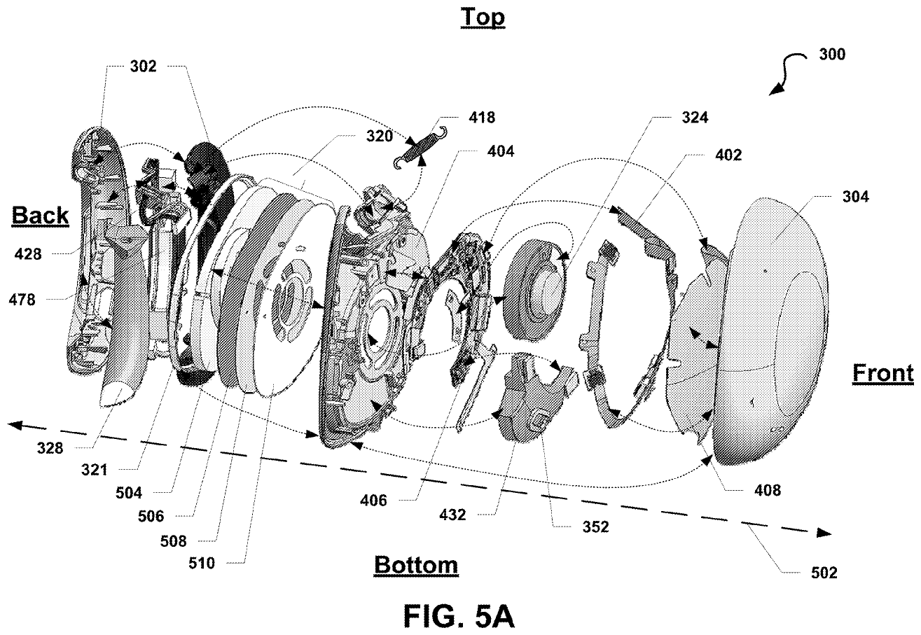

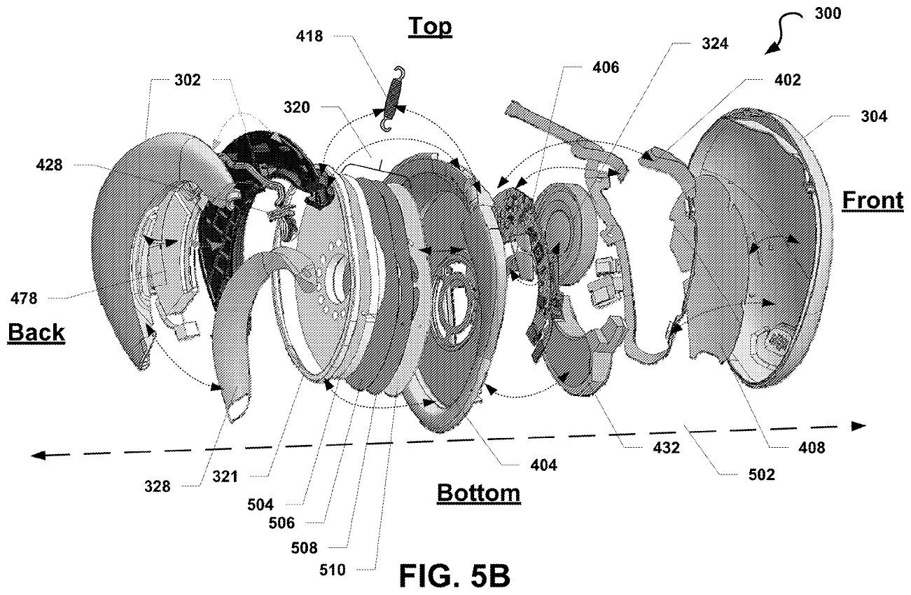

FIGS. 5A-5B illustrate different exploded views of components included in the ear-worn device 300 (e.g., as described with reference to FIGS. 3A-4J), according to some embodiments. The components of the ear-worn device 300 may generally be coupled to each other along an axis of assembly 502, as indicated by dotted arrows between components, and as further described herein.

In the examples illustrated in FIGS. 5A-5B, the electrical leads 428 may be coupled to the battery 478. The battery 478 may be housed entirely within the hooking body 302 (e.g., by placing the battery 478 between two sections of the hooking body 302), and the electrical leads 428 may be partially housed within the hooking body 302. The ear-sizing attachment 328 may be coupled to an anterior side of the hooking body 302 after the battery 478 and electrical leads 428 are positioned within the hooking body 302.

In some embodiments, the ear pad 320 may include a covering member 504, a padding member 506, an adhesive member 508, and a stiffener member 510. The covering member 504 may include or be made from one or more materials suitable for physically engaging a user's ear, such as cloth or silk. The padding member 506 may include or be made from soft materials suitable for cushioning a user's ear while the ear-worn device 300 is secured to the user's ear. The adhesive member 508 may include or be made from adhesive materials or materials that, in the presence of heat, acquire adhesive properties. The stiffener member 510 may be made from one or more of, or a combination of, at least partially rigid materials, such as plastic or rubber. The stiffener member 510 may be configured to have a flat or substantially flat front surface having a shape that is suitable for positioning within a cavity formed by the device body substrate 404.

In some embodiments, the adhesive member 508 may be coupled to (or applied to) the stiffener member 510. The padding member 506 may be coupled to the adhesive member 508. The covering member 504 may be coupled to the adhesive member 508 and/or the stiffener member 510. Once so coupled, the covering member 504, the padding member 506, the adhesive member 508, and the stiffener member 510 may be positioned within a cavity formed by the device body substrate 404 (e.g., as described above). The ring member 321 may be inserted around the covering member 504, the padding member 506, the adhesive member 508, and the stiffener member 510. The ring member 321 may be configured to hold the covering member 504, the padding member 506, the adhesive member 508, and the stiffener member 510 within the cavity of the device body 306.

The hooking body 302 may be coupled to the device body substrate 404 to form a hinge as generally described (e.g., the hinge 330 as described with reference to FIGS. 3A-4J). Once the hooking body 302 is coupled to the device body substrate 404, the spring 418 may be coupled to the hooking body 302 and the device body substrate 404. In some embodiments, an amount of torque generated by the spring 418 while so coupled may be adjusted prior to completing assembly of the ear-worn device 300 (e.g., prior to coupling the device cover 304 to the device body substrate 404).

In some embodiments, the printed circuit board 406 may be coupled to a front-facing surface of the device body substrate 404. The electrical leads 428 may be positioned to run through the hinge formed by the hooking body 302 and the device body substrate 404 and to be electrically coupled with the printed circuit board 406. The speaker housing 432 may also be coupled to a front-facing surface of the device body substrate 404. The first speaker system 324 and the second speaker system 432 included in the speaker housing 432 may be electronically coupled to the printed circuit board 406. The flex circuit board 402 may be electronically coupled to the printed circuit board 406. The touch sensor 408 may be electronically coupled to the flex circuit board 402 and coupled to the device cover 304. The device cover 304 may be coupled to the device body substrate 404. Once coupled to the device body substrate 404, the device cover 304 may, in part, form an acoustically controlled volume of air within the device body 306.

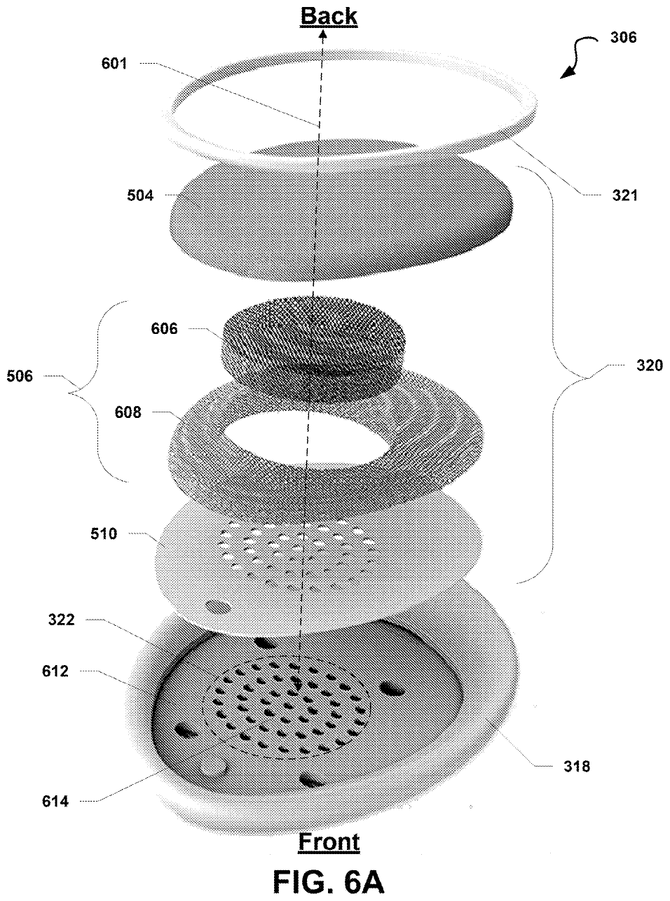

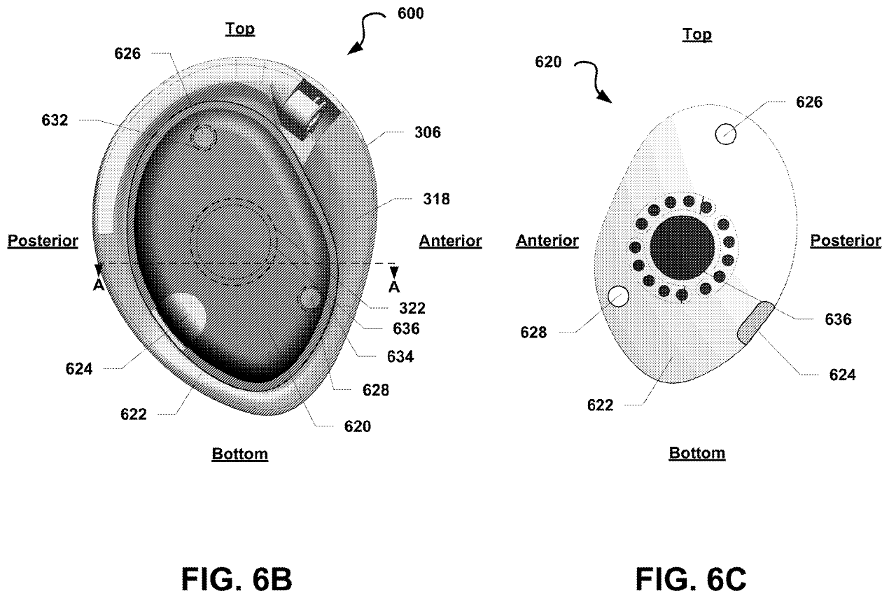

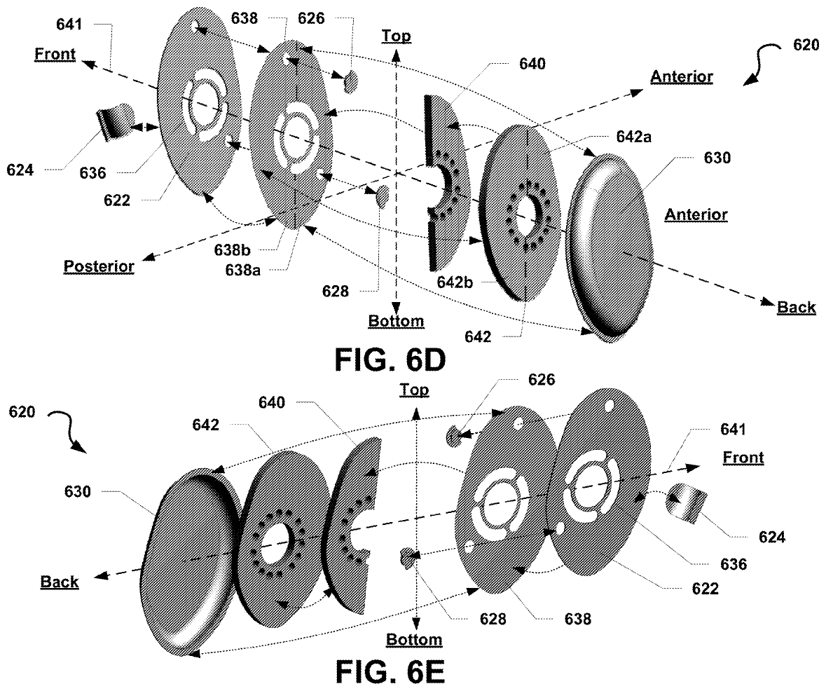

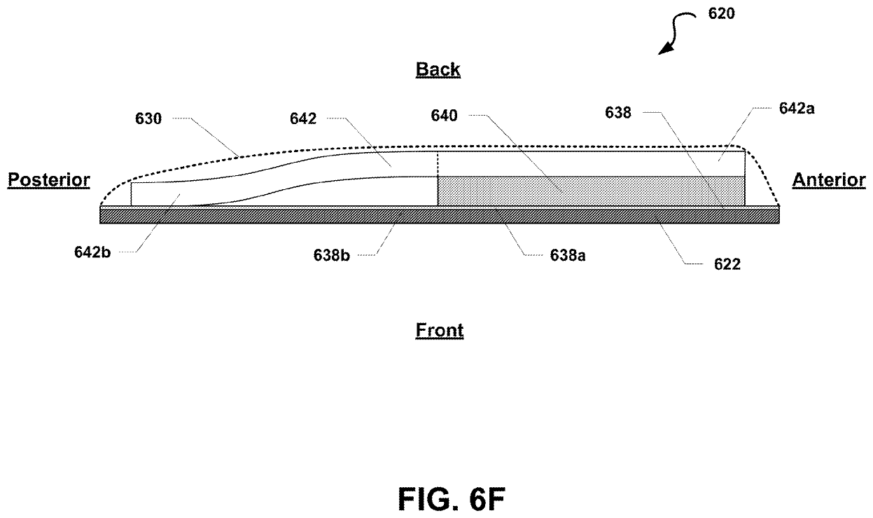

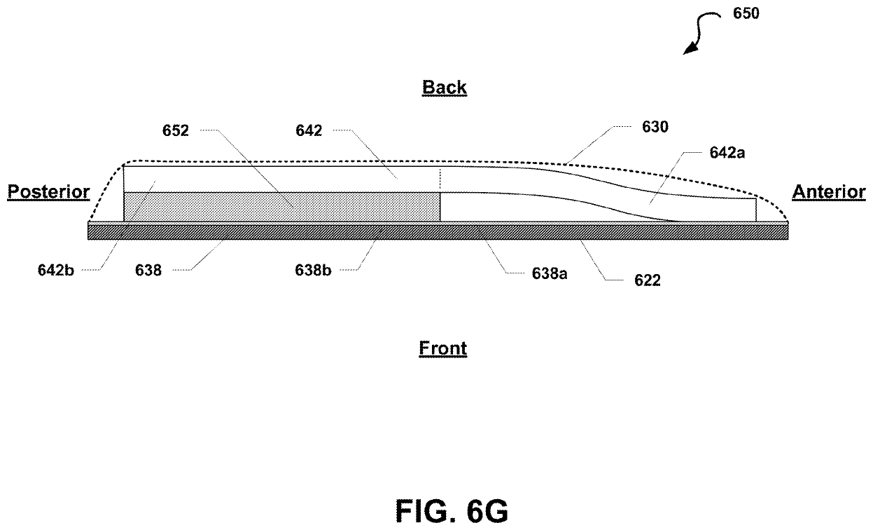

FIG. 6A illustrates a partially exploded view of the device body 306 (e.g., as described at least with reference to FIGS. 3A-5B), according to some embodiments. In the example illustrated in FIG. 6A, the ear pad 320 and the ring member 321 are illustrated in an exploded view along an axis of assembly (represented by dotted line 601). In some embodiments, the ear pad 320 may include the stiffener member 510, a first portion of acoustic material 608, a second portion of acoustic material 606, and the covering member 504. In such embodiments, the stiffener member 510 may be configured to include or be made from a relatively rigid material (e.g., a hard plastic) and may be configured to have a shape that is the same as or substantially similar to a recessed area or space formed by the edge member 318 of the device body 306 (e.g., as generally described with reference to FIGS. 3A-3L).

In some embodiments, the first portion of acoustic material 608 and the second portion of acoustic material 606 may be made from or may include relatively soft, pliable materials, such as soft foams or rubber. When the ear-worn device 300 is secured to the user's ear, the first and second portions of acoustic material 606, 608 may directly or indirectly physically engage the user's ear and may conform to various physical anatomical features of the user's ear (e.g., as generally described with reference to FIGS. 3A-3L). In some embodiments, the first portion of acoustic material 608 may be configured to include or be made from materials that are acoustically opaque so that ambient sounds (or certain frequencies of sounds) are attenuated or blocked (at least partially) from reaching the user's ear canal while the ear pad 320 is pressed against the user's ear. In some embodiments, the second portion of acoustic material 606 may be configured to include or be made from materials that are acoustically transparent so that ambient sound (or certain frequencies of sounds) easily pass through the second portion of acoustic material 606. Specifically, in such embodiments, when the ear pad 320 is assembled and coupled to a surface 612 of the ear body 306 (e.g., as further described herein), the second portion of acoustic material 606 may be positioned near or coupled to a portion of the surface 612 that includes a speaker port 614. The speaker port 614 may be configured to enable sound to be projected from the first speaker system 324 (obscured by the surface 612) through the speaker port 614 through these one or more openings. As such, when the ear pad 320 is coupled to the surface 612, the second portion of acoustic material 606 may be made from or may include acoustically transparent materials to enable the sound projected through the speaker port 614 to pass through the second portion of acoustic material 606.

In some embodiments, the first portion of acoustic material 608 may be configured to include an opening, and the second portion of acoustic material 606 may be configured to have a shape that fills the opening when the ear pad 320 is assembled along the axis 601. In some alternative embodiments, the first and second portions of acoustic material 606, 608 may formed as a single structure and may be made from or may include acoustically transparent materials.

In some embodiments, a back surface of the stiffener member 510 may be coupled to (or placed against) the surface 612 along the axis 601. A front surface of the stiffener member 510 may be coupled to a back surface of the first and second portions of acoustic material 606, 608. The covering member 504 may be positioned to cover the front surface of the first and second portions of acoustic materials 606, 608. In some embodiments, the covering member 504 may be coupled to the stiffener member 510. As described (e.g., with reference to FIGS. 3A-3L), once the ear pad 320 is assembled and coupled to the surface 612, the ring member 321 may be inserted between the ear pad 320 and the edge member 318 to hold the ear pad 320 in place. In some embodiments in which the ear pad 320 is placed against the surface 612 but not coupled to the surface 612, the ring member 321 may hold the ear pad 320 against the surface 612. The ring member 321 may be selectively removed from the device body 306 to enable the ear pad 320 to be removed from the device body 306, for example, to allow for cleaning or replacement of the ear pad 320.

FIGS. 6B-6F illustrate views of an ear pad 620 having an alternative design, according to some embodiments. Specifically, FIG. 6B illustrates an external back-side view of a partially assembled ear-worn device 600 that includes the ear pad 620. FIG. 6C illustrates a front view of the ear pad 620 when decoupled from the ear-worn device 600. FIG. 6D illustrates a perspective, exploded view of back and posterior sides of the ear pad 620. FIG. 6E illustrates a perspective, exploded view of front and anterior sides of the ear pad 620. FIG. 6F illustrates a cross-sectional view from a bottom side of the ear pad 620.

With reference to FIGS. 6B-6C, in some embodiments, the ear-worn device 600 may include the device body 306 that is coupled to the ear pad 620. The device body 306 may be configured according to one or more embodiments described herein (e.g., with reference to one or more embodiments described in relation to FIGS. 3A-5B). Additionally, while not illustrated, the ear-worn device 600 may include one or more components of and/or may be configured similarly to one or more embodiments of the ear-worn device 300 described with reference to one or more of FIGS. 3A-5B. For example, the ear-worn device 600 may include a hooking body (e.g., the hooking body 302 coupled to the device body 306) that is intentionally omitted from the example illustrated in FIG. 6B to provide an unobstructed view of the ear-pad 620. Descriptions of configurations of one or more embodiments of the ear-worn device 600 or the device body 302 that have been previously described herein (e.g., with reference to FIGS. 3A-5B) are omitted for conciseness.

The ear pad 620 may generally be configured to include pliable material suitable for engaging a user's ear while the ear-worn device 600 is secured to the user's ear (e.g., via a hooking body included in the ear-worn device 600). Specifically, when the ear-worn device 600 is secured to a user's ear, the pliable material of the ear-pad 620 may press against the user's outer ear. The pliable material may include one or more materials, or combination of materials, including foam, plastics, leather, rubber, or the like. Some configurations of the ear pad 620, according to some embodiments, are further described herein (e.g., with reference to FIGS. 6D-6F).

As described (e.g., with reference to one or more of FIGS. 3A-5B), the device body 306 may include an edge member 318 that is configured to form a cavity around at least a portion of the device body 306. In some embodiments, the ear pad 620 may be positioned within the cavity formed by the edge member 318 and may be coupled to the device body 306 while positioned within the cavity. Specifically, once the ear pad 620 is positioned within the cavity, one or more coupling devices included in the ear pad 620 may engage or otherwise couple with one or more corresponding coupling devices included on or in the device body 306. In the example illustrated in FIG. 6B, the ear pad 620 may include a first coupling device 626 and a second coupling device 628, which may be configured to couple, respectively, to a first complementary coupling device 632 and a second complementary coupling device 634 included in the device body 306. The first coupling device 626 and the second coupling device 628 may selectively couple to the first complementary coupling device 632 and the second complementary coupling device 628 to facilitate ease of removal of the ear pad 620 from the device body 306 (e.g., for cleaning or replacement purposes). In some embodiments, the coupling devices 626, 628 may include magnet elements (e.g., permanent magnets or ferromagnetic materials) that may be attracted to magnet elements included in the complementary coupling devices 632, 634.

In some embodiments, the ear pad 620 may include a decoupling member 624. The decoupling member 624 may be configured to enable a user selectively to decouple the ear pad 620 from the device body 306. The decoupling member 624 may include fabric or another pliable material configured as a pull tab. In such embodiments, when the ear pad 620 is positioned within the cavity formed by the edge member 318 of the device body 306, a user may pull the decoupling member 624 to separate the ear pad 620 from the device body 306.

With reference to the examples illustrated in FIGS. 6B-6C, the ear pad 620 may include a stiffener member 622 made from one or more of, or a combination of, at least partially rigid materials, such as plastic or rubber. As further described herein, the stiffener member 622 may be configured to provide structural support to one or more portions of padding and fabric that are coupled to the stiffener member 622. The stiffener member 622 may be configured to have a flat or substantially flat front surface having a shape that is suitable for positioning within a cavity formed by the edge member 318 of the device body 306 (e.g., as described above). The stiffener member 622 may physically engage a surface of the device body 306 when the ear pad 620 is positioned within the cavity and/or when the ear pad 620 is otherwise coupled to the device body 306. In some embodiments, the first coupling device 626 and second coupling device 628 may be coupled to or included in the stiffener member 622 so that when the front surface of the stiffener member 622 engages the device body 306, the first coupling device 626 and the second coupling device 628 may couple with the first complementary coupling device 632 and the second complementary coupling device 634, respectively.

In some embodiments, the stiffener member 622 may include an opening 636. When the ear pad 620 is coupled to the device body 306 (e.g., as described above), the opening 636 may be positioned near or adjacent to the mid-ear portion 322 of the device body 306. In such embodiments, the opening 636 may enable sound emitted from the mid-ear portion 322 of the device body 306 (e.g., via the first speaker system 324, not shown) to pass through the stiffener member 622. As a result, while the ear-worn device 600 is secured to a user, sound generated by the ear-worn device 600 may reach the user without being attenuated (or only slightly attenuated) by the ear-pad 620. In some additional (or alternative) embodiments, the stiffener member 622 may be configured to have a shape in at least one dimension that corresponds to a shape of the cavity formed by the edge member 318 so that the stiffener member 622 may be positioned within the cavity when coupling the ear pad 620 to the device body 306.

With reference to FIGS. 6D-6E, according to some embodiments, the ear pad 620 may include the stiffener member 622, an optional adhesive member 638, the first coupling device 626, the second coupling device 628, a first padding member 640, a second padding member 642, and a covering member 630. As described, the stiffener member 622 may be configured to include or be made from a relatively rigid material (e.g., a hard plastic) and may be configured to have a shape that is the same as or substantially similar to a recessed area or space formed by the edge member 318 of the device body 306. In some embodiments, the optional adhesive member 638 may be made from or may include one or more materials that, while heated, acquires adhesive properties (e.g., a hot melt film), as would be known by one skilled in the art.

Each of the first padding member 640 and the second padding member 642 may be made from or may include relatively soft, pliable materials, such as soft foams or rubber. Specifically, the first and second padding members 640, 642 may directly or indirectly physically engage the user's ear and may conform to various physical anatomical features of the user's ear (e.g., as generally described with reference to FIGS. 6B-6C).

In some embodiments (e.g., as depicted in the examples illustrated in FIGS. 6D-6E), the stiffener member 622, the adhesive layer 638, the first padding member 640, the second padding member 642, and the covering member 630 may be coupled together along an axis of assembly (e.g., along dotted line 641). The decoupling member 624 may be coupled to a surface of the stiffener member 624. A back surface of the stiffener member 622 may be coupled to a front surface of the adhesive layer 638. The adhesive layer 638 may be configured to couple one or more other components of the ear pad 620 to the stiffener member 622.

In some embodiments, each of the coupling devices 626 and 628 may include a protruding portion and a flange portion. The protruding portions the coupling devices 626 and 628 may be inserted through aligning openings in the stiffener member 622 and the adhesive layer 638. Front sides of the flange portions of the coupling devices 626 and 628 may be coupled to a back side of the adhesive layer 638 near the respectively openings in the adhesive layer 638 through which the protruding portions of the coupling devices 626, 628 are inserted.

A front side of the first padding member 640 may be coupled to a back side of a portion of the adhesive member near an anterior side of the adhesive member 638 (e.g., an anterior portion 638a of the adhesive member 638). A back side of the first padding member 640 may be coupled to a front side of a portion of the second padding member 642 near an anterior side of the second padding member 642 (e.g., an anterior portion 642a of the second padding member 638). In some embodiments, a front side of a portion of the second padding member 642 near a posterior side of the second padding member 642 (e.g., a posterior portion 642b of the second padding member 642) may be coupled directly to a portion of the adhesive member 638 near a posterior side of the adhesive member 638 (e.g., a posterior portion 638b of the adhesive member 638). In some embodiments, the first padding member 640 may be configured to have a shape of the posterior portion 642b of the second padding member 642.

In some embodiments, the adhesive member 638 may have one or more dimensions and/or shapes that are the same or substantially similar to the stiffener member 622. Similarly, at least the second padding member 642 may be configured to have at least one or more dimensions and/or shapes that are the same or substantially similar to the stiffener member 622. By way of a non-limiting example, the adhesive member 638 may, when coupled to the stiffener member 622, cover all or substantially all of a back surface of the stiffener member 622. In some embodiments, a shape of the adhesive member 638 and the second padding member 642 may be the same or substantially the same as a shape of the stiffener member 622. In some further embodiments, the second padding member 642 may have a shape that is scaled down from a shape of the stiffener member 622 (e.g., as illustrated at least in FIG. 6F). In some embodiments, the first padding member 640 may be configured to have a shape and one or more dimensions that are the same or substantially the same as the anterior portion 642a of the second padding member 642.

The covering member 630 may include or be made from one or more materials, or a combination of such materials, that may be suitable for direct contact with a user's ear. By way of a non-limiting example, the covering member 630 may include cloth, leather, or soft synthetic fibers. In some embodiments, the covering member 630 may be flexible and configurable to cover one or more of the padding members 640, 642. For example, when the padding members 640, 642 are coupled to the adhesive member 638, an edge of the covering member 630 may be coupled to a back side of the adhesive member 638 so that the covering member 630 at least substantially covers a back side of the second padding member 642.

In some embodiments, the adhesive member 638, the first padding member 640, and the second padding member 642 may each include at least one opening such that, when the ear pad 620 is assembled, the openings of the adhesive member 638, the first padding member 640, and the second padding member 642 may align, at least substantially, with the mid-ear portion 636 of the stiffener member 622. In such embodiments, when the ear pad 620 is coupled to the device body 306, sound generated by the device body 306 (e.g., via the first speaker system 324) may pass through the ear pad 620 without significant distortion or attenuation. In some embodiments, the covering member 630 may be made from one or more acoustically transparent materials to enable such sound to pass through the covering member 630 without significant distortion or attenuation.

In some alternative embodiments, the optional adhesive member 638 may be excluded from the ear pad 620. In such embodiments, coupling devices 626, 628, the first padding member 640, second padding member 642, and covering member 630 may be coupled directly to the stiffener member 622 (e.g., via conventional adhesives) rather than indirectly via the adhesive member 638.

FIG. 6F illustrates a cross-sectional view of the ear pad 620 taken alone cross-sectional line A-A (as depicted in FIG. 6B). As described (e.g., with reference to FIGS. 6D-6E), the anterior portion 642a of the second padding member 642 may be coupled to the first padding member 640, which may in turn be coupled to the adhesive member 622. In some embodiments, because the posterior portion 642b of the second padding member 642 is coupled directly to the adhesive member 622, the ear pad 620 may have a higher profile towards an anterior side of the ear pad 620 compared with a profile closer to a posterior side of the ear pad 620. As a result, when the ear pad 620 is coupled to the device body 306 and the ear worn-device 600 is secured to an ear of a user, the ear pad 620 may protrude less towards a posterior side ear than the extent to which the ear pad 620 protrudes towards an anterior side of the user's ear. Typically, the outer portion of a human ear is more pronounced towards a posterior side of the ear and less pronounced towards an anterior side of the ear. Accordingly, the ear pad 620 may engage the user's ear more evenly across both anterior and posterior portions of the user's ear by protruding to a lesser extent towards the posterior portion of a user's ear and protruding to a greater extent towards the anterior portion of the user's ear.

FIG. 6G illustrates a cross-sectional view from a bottom side of an ear pad 650, according to some alternative embodiments. The ear pad 650 may include one or more components configured as described above with reference to the ear pad 620 (e.g., as described above with reference to at least one of FIGS. 6B-6F). By way of a non-limiting example, the ear pad 650 may include the stiffener member 622, the adhesive member 638, the second padding member 642, and the covering member 630.

In some embodiments, the ear pad 650 may include a first padding member 652. The first padding member 652 may be configured to have a shape of the posterior portion 642b of the second padding member 642. A front side of the first padding member 652 may be coupled to a back side of the posterior portion 638b of the adhesive member 638. A back side of the first padding member 652 may be coupled to a front side of the posterior portion 642b of the second padding member 638). In some embodiments, a front side of the anterior portion 642a of the second padding member 642 may at least partially be coupled to the anterior portion 638a of the adhesive member 638. Because the anterior portion 642a of the second padding member 642 is coupled directly to the adhesive member 622, the ear pad 650 may have a higher profile towards a posterior side of the ear pad 650 compared with a profile closer to an anterior side of the ear pad 650. As a result, when the ear pad 650 is coupled to the device body 306 and the ear worn-device 600 is secured to an ear of a user, the ear pad 650 may protrude less towards an anterior side of the ear than the extent to which the ear pad 650 protrudes towards a posterior side of the user's ear. Accordingly, the ear pad 650 may provide additional padding on the posterior portion of the user's ear, which may increase the user's comfort.

FIGS. 7A and 7B illustrates views of a hooking body 701 and an ear-fitting attachment 703, according to some alternative embodiments. Specifically, FIG. 7A illustrates an external view of a back side of the hooking body 701 and the ear-fitting attachment 703 in which the ear-fitting attachment 703 is decoupled from the hooking body 701. FIG. 7B illustrates a cross-sectional view of the hooking body 701 and the ear-fitting attachment 703 taken along a cross-sectional line B-B depicted in FIG. 7A. In some embodiments, the hooking body 701 may be configured to be couplable to a device body as part of an ear-worn device (e.g., as generally described with reference to the hooking body 302, the device body 306, and the ear-worn device 300 illustrated in FIGS. 3A-5B). Accordingly, in some embodiments (not shown), the hooking body 701 may be configured to secure such an ear-worn device to a user's ear as generally described in relation to various embodiments described herein.

The ear-fitting attachment 703 may be configured to be selectively coupled to the hooking body 701. In some embodiments illustrated in FIGS. 7A-7B, the hooking body 701 and the ear-fitting attachment 703 may be configured to include corresponding coupling portions. Specifically, in such embodiments, the hooking body 701 may be configured to include one or more coupling devices (e.g., a male coupling portion 702), and the ear-fitting attachment 703 may be configured to include one or more corresponding coupling devices (e.g., a corresponding female coupling portion 704). In the example illustrated in FIGS. 7A-7B, the male coupling portion 702 may be positioned towards an anterior side of the hooking body 701, whereas the female coupling portion 704 may be positioned towards the posterior side of the ear-fitting attachment 703. In some embodiments, the male coupling portion 702 may be configured as one or more protrusions from the hooking body 701, and the female coupling portion 704 may be configured as one or more recesses in the ear-fitting attachment 703. In such embodiments, the hooking body 701 and the ear-fitting attachment 703 may be coupled together by inserting the male coupling portion 702 protruding from the hooking body 701 into the one or more recesses formed in the ear-fitting attachment 703. Once coupled together, the hooking body 701 and the ear-fitting attachment 703 may remain coupled together through friction and/or one or more coupling device (not show), such as reciprocal magnets, snaps, or the like. In some alternative embodiments, the hooking body 701 may instead include a female coupling portion and the ear-fitting attachment 703 may include a male coupling portion.

The ear-fitting attachment 703 may be configured to engage a surface of a root of a posterior portion of a user's ear when the ear-fitting attachment 703 is coupled to the hooking body 701 and the hooking body 701 is secured to the user's ear. In some embodiments, because the ear-fitting attachment 703 and the hooking body 701 are selectively couplable, the ear-fitting attachment 703 may be replaceable with another ear-fitting attachment that has a different cross-sectional profile that may be more suitable for a particular user's ear. For example, the cross-sectional profile may be wider or narrower or have various other shapes to accommodate the unique shape of a user's ear. Accordingly, a user may couple one of numerous ear-fitting attachments 703 of different sizes and/or shapes to the hooking body 701 based on the user's preference.

FIGS. 7C-7K illustrates various views of an ear-fitting attachment 710, according to some embodiments. FIG. 7C illustrates an external view of a back side of the ear-fitting attachment 710 when the ear-fitting attachment 710 is coupled to a hooking body 302, and FIG. 7D illustrates an external view of a back side of the ear-fitting attachment 710 while the ear-fitting attachment 710 is decoupled from the hooking body 302. FIG. 7E illustrates an exterior view of an anterior side of the ear-fitting attachment 710. FIG. 7F illustrates an exterior view of a posterior side of the ear-fitting attachment 710. FIG. 7G illustrates an exterior view of a front side of the ear-fitting attachment 710. FIG. 7H illustrates a perspective view of back and anterior sides of the ear-fitting attachment 710.

With reference to FIGS. 7C-7H, the hooking body 302 may be configured to couple with a device body via a hinge (not shown) as part of an ear-worn device as generally described herein (e.g., the ear-worn device 300 as described with reference to FIGS. 3A-5B). Some duplicative descriptions of the hooking body 302 are not repeated for conciseness. In some embodiments, the hooking body 302 may include one or more coupling devices configured to enable the hooking body 302 to couple with the ear-fitting attachment 710. In the examples illustrated in FIGS. 7C-7D, the hooking body 302 may be configured to include the coupling devices 474a-474c. As described (e.g., with reference to FIGS. 4A-5B), the coupling devices 474a-474c may be configured to couple selectively with one or more coupling devices included on an ear-fitting attachment. As illustrated (e.g., in FIGS. 7E-7H), the ear-fitting attachment 710 may include one or more coupling devices (e.g., coupling devices 714, 716, 720), configured to be coupleable with the coupling devices 474a-474c of the hooking body 302. By way of a non-limiting example, the coupling devices 474b, 474c of the hooking body 302 may be configured as male coupling devices (e.g., a protruding hard cap) that may be coupled to the coupling devices 716, 720 of the ear-fitting attachment 710. In this example, the coupling devices 716, 720 may be configured as flexible recesses that enable the hard caps of the coupling devices 716, 720 to pass through a narrow channel into a cavity that houses the hard caps of the coupling devices 474b, 474c when the coupling devices 474b, 474c are coupled to the coupling devices 716, 720.

In some embodiments, one or more coupling devices of the ear-fitting attachment 710 may be configured to maintain alignment of the ear-fitting attachment to the hooking body 302 along an anterior side of the hooking body 302. In the examples illustrated in FIGS. 7E-7H, the coupling device 474a of the hooking body 302 may be a cylindrical protrusion, and the coupling device 714 of the ear-fitting attachment 710 may be a cylindrically shaped recess. When the hooking body 302 is coupled to the ear-fitting attachment 710, the coupling device 474a may be inserted into the coupling device 714, thereby facilitating alignment of the ear-fitting attachment 710 along an anterior side of the hooking body 302. In some embodiments in which the hooking body 302 includes the protruding portion 494 (e.g., as generally described with reference to at least one of FIGS. 4A-5B), the ear-fitting attachment 710 may include a recessed area 718 that may accommodate the protruding portion 494 of the hooking body 302 when the hooking body 302 and the ear-fitting attachment 710 are coupled. In such embodiments, while the protruding portion 494 is positioned within the recessed area 718, the recessed area 718 may also facilitate alignment of the ear-fitting attachment 710 along an anterior side of the hooking body 302.

In some embodiments, the hooking body 302 and the coupling device 710 may be configured so that, when the hooking body 302 and the coupling device 710 are coupled together, external surfaces of the coupling device 710 and the hooking body 302 may be at least substantially continuous. By way of a non-limiting example, when the hooking body 302 and the coupling device 710 are coupled together, an external surface of the hooking body 302 may smoothly transition to an external surface of the ear-fitting attachment 710 towards an anterior side of the hooking body 302. In such embodiments, due to this smooth transition, the ear-fitting attachment 710 and the hooking body 302 may be or appear to be contiguous.

In some embodiments, a profile shape of the ear-fitting attachment 710, when viewed from a front or back side of the ear-fitting attachment 710 may be substantially curved to approximate a curvature of a root of a posterior portion of a human ear. By way of a non-limiting example illustrated in at least FIG. 7D, a shape of an anterior surface 712 of the ear-fitting attachment 710 is substantially curved in a shape of a “C.” As generally described (e.g., with reference the ear-fitting attachment 328 depicted in at least one of FIGS. 3A-5B), when an ear-fitting attachment is coupled to a hooking body included in an ear-worn device, the anterior surface 712 of the ear-fitting attachment engages a root of a posterior portion of a user's ear when the ear-worn device is secured to the user's ear. The anterior surface 712 of the ear-fitting attachment 710 may be configured to ensure that the user does not experience hard edges that apply pressure and cause discomfort. In some embodiments, the ear-fitting attachment 710 may be configured such that the anterior surface 712 of ear-fitting attachment 710 is curved along a cross-sectional profile so that the anterior surface 712 of the ear-fitting attachment 710 fits snuggly against a root of the posterior portion of the user's ear.

FIGS. 7I-7K illustrate cross-sectional views of the anterior surface 712 of the ear-fitting attachment 710, according to some embodiments. FIG. 7I illustrates a cross-sectional view of the ear-fitting attachment 710 along a cross-sectional line C-C as depicted in FIG. 7D. FIG. 7J illustrates a cross-sectional view of the ear-fitting attachment 710 along a cross-sectional line D-D as depicted in FIG. 7D. FIG. 7I illustrates a cross-sectional view of the ear-fitting attachment 710 along a cross-sectional line E-E as depicted in FIG. 7D.

With reference to FIGS. 7D and 7I-7K, a cross-sectional shape of the anterior surface 712 of the ear-fitting attachment 710 (taken along cross-sectional line C-C depicted in FIG. 7D) may be substantially curved (e.g., “C” shaped) as the anterior surface 712 nears an anterior side of the ear-fitting attachment 710. In some embodiments (e.g., as illustrated in FIG. 7I), at the further extent to which the anterior surface 712 extends toward an anterior side of the ear-fitting attachment 710, the anterior surface 712 may terminate at a tangent point along a front-to-back axis (e.g., as represented by dotted line 718). As the anterior surface 712 transitions from a top side to a bottom side of the ear-fitting attachment 710, the cross-sectional shape of the anterior surface 712 may transition from an at least substantially curved shape to an at least substantially flat shape (e.g., a flat-bottomed “U” shape). In the example illustrated in FIG. 7J, a cross-sectional shape of the anterior surface 712 (taken along cross-sectional line D-D depicted in FIG. 7D) may flatten, at least substantially, as the anterior surface 712 nears an anterior side of the ear-fitting attachment 710. In some embodiments, at the further extent to which the anterior surface 712 extends toward an anterior side of the ear-fitting attachment 710, the anterior surface 712 may terminate at a line along a front-to-back axis (e.g., as represented by dotted line 720). As the anterior surface 712 continues transitioning towards a bottom side of the ear-fitting attachment 710, the cross-sectional shape of the anterior surface 712 near an anterior side of the ear-fitting attachment may transition from an at least substantially flat shape to an at least substantially curved shape (e.g., a “C” shape). In the example illustrated in FIG. 7K, a cross-sectional shape of the anterior surface 712 (taken along cross-sectional line E-E depicted in FIG. 7D) may become more curved, at least substantially, as the anterior surface 712 nears an anterior side of the ear-fitting attachment 710. In some embodiments, at the further extent to which the anterior surface 712 extends toward an anterior side of the ear-fitting attachment 710, the anterior surface 712 may terminate at a tangent point along a front-to-back axis (e.g., as represented by dotted line 724). In some embodiments, by configuring the anterior surface 712 of the ear-fitting attachment to have a cross-sectional shape that transitions (from a top side to a bottom side of the anterior surface 712) from a curved shape to a flattened shape and back to a curved shape, the anterior surface 712 of the ear-fitting attachment 710 may more accurately follow the anatomy of a root of a posterior portion of a user's ear, which may improve comfort, security, and overall user experience.

FIGS. 8A and 8B illustrates exterior views of an audio system 801 that include the ear-worn device 300 and another ear-worn device 800. FIG. 8A illustrates a front view of the audio system 801, and FIG. 8B illustrates a back view of the audio system 801. The ear-worn device 300 may be configured according to various embodiments previously described herein (e.g., with reference to FIGS. 3A-5B). With reference to FIGS. 8A-8B, the ear-worn device 800 may be configured as a mirror-image of the ear-worn device 300. In some embodiments, the ear-worn device 800 may include, but is not limited to including: a hooking body 802, a device body 806, a hinge 812, a charging connector 842, a device cover 814, an edge member 808, microphones 818, 822, 824, a lighting element 820, openings 828, 832, 834, lighting port 830, a speaker system 816, and a speaker port 826. In some embodiments, the above elements of the ear-worn device 800 may be configured as mirror images of the hooking body 302, the device body 306, the hinge 330, the charging connector 342, the device cover 304, the edge member 318, the microphones 340, 342, 344, the lighting element 350, the openings 308, 310, 314, the lighting port 312, the second speaker system 352, and the speaker port 354 of the ear-worn device 300, respectively. For ease of description, duplicative descriptions of such elements are omitted. In some embodiments (not shown), the ear-worn device 800 may include one or more other features or components that are configured as mirror images of features or components of the ear-worn device 800, including but not limited to, a processing unit, ear pad, ear-fitting attachment, a speaker system configured to project sound through the ear pad, or various other elements or features similar to those described as being included or coupled to the ear-worn device 300 (e.g., as described with reference to FIGS. 3A-5B).

The ear-worn devices 300, 800 may be configured to be coupleable together. In some embodiments, the ear-worn devices 300, 800 may be configured to include one or more coupling devices in their respective hooking bodies 302, 802. Specifically, in the example illustrated in FIG. 8B, the hooking body 302 may include or be coupled to a first coupling device 870 positioned near a top of the hooking body 302 and a second coupling device 880 positioned near a bottom of the hooking body 302. Similarly, the hooking body 802 may include or be coupled to a third coupling device 872 positioned near a top of the hooking body 802 and a fourth coupling device 882 positioned near a bottom of the hooking body 802. The ear-worn devices 300, 800 may be coupled together by causing the first and third coupling devices 870, 872 to engage and/or by causing the second and fourth coupling devices 880, 882 to engage. The coupling devices 870, 872, 880, 882 may be one or more (or a combination of) fasteners, magnets, snaps, or the like. By way of a non-limiting example, the coupling devices 870, 872, 880, 882 may be magnets, whereby at least the first coupling device 870 has a different magnetic polarity from the third coupling device 872 and the second coupling device 880 has a different magnetic polarity from the fourth coupling device 882. One or more other coupling devices may be utilized to couple the ear-worn devices 300, 800 together. The coupling devices 870, 872, 880, 882 may also be configured to allow the ear-worn devices 300, 800 to be decoupled, for example, when the ear-worn devices 300, 800 are pulled apart (e.g., along different directions of a referential line 890).

In some embodiments, the ear-worn device 300, 800 may be in electronic communication with each other (e.g., via a wireless communication signal, such as Bluetooth or near-field magnetic induction). In such embodiments, respective processing units (not show) of the ear-worn devices 300, 800 may coordinate in order to play out synchronized sound through the speaker systems 352, 816. For example, the speaker systems 316, 816 may play out music or other sounds at volumes that may be heard by nearby listeners (e.g., in the same room, house, or the like).

In some embodiments, the ear-worn device 300, 800 may, respectively, include sensors 820, 822. Each of the sensors 820, 822 may be configured to detect the presence of the other sensor or another element. The sensors 820, 822 may be in communication with a processing unit on their respective ear-worn devices 300, 800. In some embodiments, when the sensors 820, 822 detect each other (or another element in the other ear-worn device), the sensors 820, 822 may send a signal indicating that the ear-worn devices 300, 800 are coupled together. In response, the processing units may selectively deactivate features or components on their respective ear-worn devices 300, 800, such as the speaker systems 352, 816. For example, the speaker systems 352, 816 may be playing out sound while the ear-worn devices 300, 800 are not coupled together (e.g., when the sensors 820, 822 do not detect the presence of each other), but the processing units may cause the speaker systems 352, 816 to pause/stop playing out sound when the ear-worn devices 300, 800 are coupled together (e.g., when the sensors 820, 822 do detect the presence of each other). In some embodiments, the processing units may selective activate features or components on their respective ear-worn devices 300, 800 when the sensors 820, 822 do not detect the presence of each other. By way of a non-limiting example, the ear-worn devices 300, 800 may be in a low-power or “standby” state while they are coupled to each other, but upon decoupling, the processing units may activate or resume operations, activities, functions, features, etc. For example, in response to determining that the sensors 820, 822 no longer detect each other, the processing units may resume communications with each other (and/or another electronic device) and may resume playing out sound via the speaker system 324 in ear-worn device 300 and a similar situated speaker system (not visible) in ear-worn device 800.

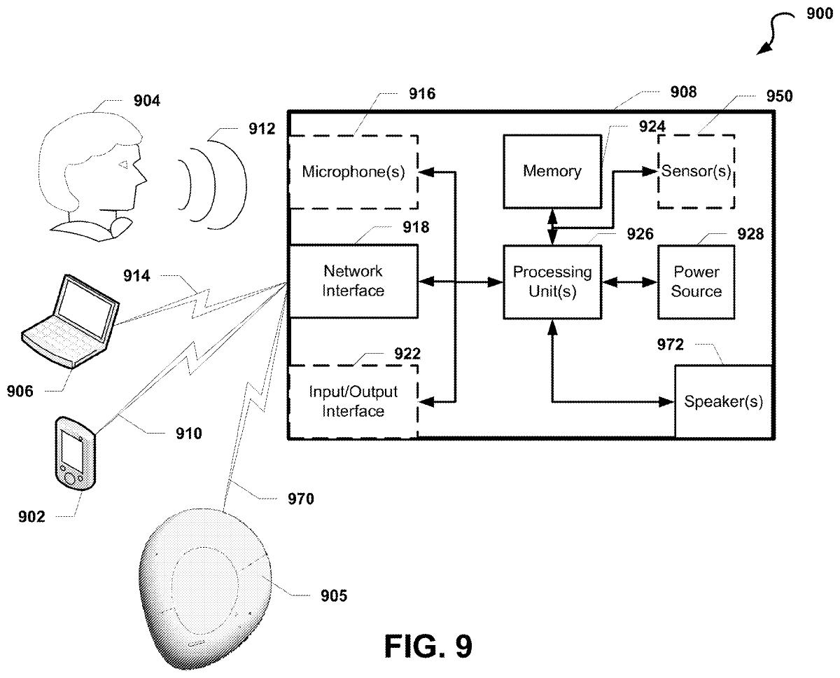

FIG. 9 is a functional block diagram of an illustrative operating environment 900 suitable for implementing aspects of the present disclosure. The operating environment 900 includes an ear-worn device 908 that may be configured to receive audio data from various sources, including a mobile computing device 902, an analog source of sound 904 (e.g., a human), or another computing device 906.

The example illustrated in FIG. 9 depicts a general architecture of the ear-worn device 908 that may be configured to playout audio, among other functions. The general architecture of the ear-worn device 908 includes an arrangement of computer hardware and/or software components. The ear-worn device 908 may include more (or fewer) elements than those shown in FIG. 9. It is not necessary, however, that all of these generally conventional elements be shown in order to provide an enabling disclosure. In some embodiments, the ear-worn device 908 may be configured as described above with reference to the ear-worn device 100 (e.g., described with reference to FIG. 1A-2C) and/or the ear-worn devices 300, 600, 800 (e.g., as described with reference to any of FIGS. 3A-8B).

As illustrated, the ear-worn device 908 may include an input/output device interface 922, a network interface 918, one or more microphones 916, a memory 924, one or more processing units 926, a power source 928, and one or more speakers 932, all of which may communicate with one another by way of a communication bus. The network interface 918 may provide connectivity to one or more networks or computing systems, and the processing unit 926 may receive and/or send information and instructions from/to other computing systems or services via the network interface 918. In some embodiments, the network interface 918 may be configured to communicate with the mobile computing device 902 and/or the other computing device 906 via wireless communication links 910 and 914, such as via a Wi-Fi Direct or Bluetooth communication links. The network interface 918 may also (or alternatively) be configured to communicate with the computing devices 902 and 906 via a wired communication link (not shown). Those skilled in the art will recognize that the computing devices 902 and 906 may be any of a number of computing devices capable of communicating via a wireless or wired link including, but not limited to, a laptop, personal computer, personal digital assistant (PDA), hybrid PDA/mobile phone, mobile phone, smartphone, wearable computing device (e.g., wireless headphones or earphones), electronic book reader, digital media player, tablet computer, gaming console or controller, kiosk, augmented or virtual reality device, other wireless device, set-top or other television box, or the like. In such embodiments, the network interface 918 may receive audio data from the mobile computing devices 902 and/or 906 and may provide the audio data to the processing unit 926. In such embodiments, the processing unit 926 may cause the audio data to be transformed into an electrical audio signal that is provided to the speaker 932 for output as sound. In some embodiments, the network interface 918 may provide connectivity to another ear-worn device 905, such as via a wireless communication link 913. In such embodiments, the other ear-worn device 905 may be configured as a mirror image of the ear-worn device 908.

The one or more processing units 926 may communicate to and from memory 924. In some embodiments, the memory 924 may include RAM, ROM, and/or other persistent, auxiliary or non-transitory computer-readable media. The memory 924 may store an operating system that provides computer program instructions for use by the one or more processing units 926 in the general administration and operation of the ear-worn device 908. In some embodiments, the memory 924 may contain digital representations of audio data or electronic audio signals (e.g., digital copies of songs or videos with audio). In such embodiments, the one or more processing units 926 may obtain the audio data or electronic audio signals from the memory 924 and may provide electronic audio signals to the one or more speakers 932 for playout as sound.

In some embodiments, the input/output interface 922 may also receive input from an input device (not shown), such as a keyboard, mouse, digital pen, microphone, touch screen, gesture recognition system, voice recognition system, image recognition through an imaging device (which may capture eye, hand, head, body tracking data and/or placement), gamepad, accelerometer, gyroscope, or another input device known in the art. In some embodiments, the one or more microphones 916 may be configured to receive sound 930 from an analog sound source 904. For example, the one or more microphones 916 may be configured to receive human speech. The one or more microphones 916 may further be configured to convert the sound into audio data or electrical audio signals that are directly or indirectly provided to the one or more speakers 932 for output as sound.

Each of the communication links 910 and 914 may be communication paths through networks (not shown), which may include wired networks, wireless networks or combination thereof. In addition, such networks may be personal area networks, local area networks, wide area networks, cable networks, satellite networks, cellular telephone networks, etc. or combination thereof. In addition, the networks may be a personal area network, local area network, wide area network, over-the-air broadcast network (e.g., for radio or television), cable network, satellite network, cellular telephone network, or combination thereof. In some embodiments, the networks may be private or semi-private networks, such as a corporate or university intranets. The networks may also include one or more wireless networks, such as a Global System for Mobile Communications (GSM) network, a Code Division Multiple Access (CDMA) network, a Long Term Evolution (LTE) network, or some other type of wireless network. Protocols and components for communicating via the Internet or any of the other aforementioned types of communication networks are well known to those skilled in the art and, thus, are not described in more detail herein.

In some embodiments, the ear-worn device 908 may include one or more sensors 950. The one or more sensors 950 may include, but are not limited to, one or more biometric sensors, heat sensors, gyroscopic sensors, accelerometers, pressure sensors, force sensors, light sensors, or the like. In such embodiment, the one or more sensors 950 may be configured to obtain sensor information from a user of the ear-worn device 908 and/or from an environment in which the ear-worn device 908 is worn by the user. The one or more processing units 926 may receive sensor readings from the one or more sensors 950 and may generate one or more outputs based on these sensor readings. For example, the processing unit 926 may configure a light-emitting diode included on the ear-worn device (not shown) to flash according to a preconfigured patterned based on the sensor readings.

Examples illustrated in the accompanying drawings may depict one or more embodiments of the ear-worn device as being configured for use with a left ear of a user. However, any descriptions or illustrations of the foregoing ear-worn devices that cause the ear-worn device to be suitable for use with a left ear of a user are made merely for ease of description. As such, unless otherwise limited by the claims, there is no requirement that the ear-worn devices described above must be configured for use only with left ears. For example, without loss of generality, any of the above ear-worn devices may be configured to couple to a right ear of a user by mirroring the structures described and illustrated above. Further, in some embodiments (not shown), any of the various ear-worn devices described above may be utilized with a mirrored ear-worn device that is configured to function on an opposite ear. In such embodiments, an ear-worn device and a mirrored ear-worn device may operate together as part of a single audio system because the ear-worn device may be configured for use with one ear of the user, and the mirrored ear-worn device may be coupled for use with the other ear (or vice versa). For example, in some embodiments in which the ear-worn device and the mirrored ear-worn device are configured as wearable audio systems, the user may utilize the ear-worn device and the mirrored ear-worn device together to experience stereophonic sounds in which an audio source is played simultaneously or nearly simultaneously through both the ear-worn device and the mirrored ear-worn device.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Conjunctive language such as the phrase “at least one of X, Y, and Z,” unless specifically stated otherwise, is otherwise understood with the context as used in general to present that an item, term, etc., may be either X, Y, or Z, or any combination thereof (e.g., X, Y, and/or Z). Thus, such language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

It should be emphasized that many variations and modifications may be made to the above-described embodiments, the elements of which are to be understood as being among other acceptable examples. All such modifications and variations are intended to be included herein within the scope of this disclosure and protected by the following claims.

1. An ear-worn device for coupling to an ear of a user, comprising:
a device body;
a hooking body;
an ear pad couplable to the device body; and
a hinge coupled to the device body and the hooking body;
wherein:
the hinge is configured to enable the device body to move towards and away from the hooking body between an open configuration and a closed configuration;
the device body comprises an edge member,
the edge member is configured to define a recessed area on the device body, and
the ear pad is positioned within the recessed area when coupled to the device body.

2. The ear-worn device of claim 1, wherein:
the ear pad comprises:
a stiffener member,
a first padding member, and
a second padding member comprising a posterior portion and an anterior portion;
a front surface of the anterior portion of the second padding member is coupled to a back surface of the first padding member;
a front surface of the posterior portion of the second padding member is coupled to a back surface of the stiffener member; and
a front surface of the first padding member is coupled to the back surface of the stiffener member.

3. (canceled)

4. (canceled)

5. The ear-worn device of claim 1, wherein:
the ear pad comprises:
a stiffener member comprising at least one rigid material,
a first padding member, and
a second padding member comprising a posterior portion and an anterior portion;
a front surface of the posterior portion of the second padding member is coupled to a back surface of the first padding member;
a front surface of the anterior portion of the second padding member is coupled to a back surface of the stiffener member; and
a front surface of the first padding member is coupled to the back surface of the stiffener member.

6. (canceled)

7. The ear-worn device of claim 1, wherein:
the device body comprises a first coupling device positioned towards a surface of the recessed area;
the ear pad comprises a second coupling device positioned towards a front surface of the ear pad; and
the first coupling device is coupled to the second coupling device when the ear pad is coupled to the device body.

8. The ear-worn device of claim 1, wherein the hooking body and the device body, when the ear-worn device is configured in an open configuration, form a space suitable for receiving at least a posterior portion of a human ear.

9. (canceled)

10. The ear-worn device of claim 1, further comprising a ring member configured to:
have a shape at least substantially similar to an edge of the ear pad; and
secure the ear pad to the device body within the recessed area.

11. The ear-worn device of claim 1, further comprising an ear-fitting attachment having a profile shape that accommodates at least a shape of a root of a posterior portion of a human ear.

12. (canceled)

13. The ear-worn device of claim 11, wherein the ear-fitting attachment is configured such that:
a cross-sectional shape of a first portion of an anterior surface of the ear-fitting attachment is substantially curved;
a cross-sectional shape of a second portion of the anterior surface of the ear-fitting attachment is substantially flat; and
the first portion of the anterior surface transitions into the second portion of the anterior surface.

14. The ear-worn device of claim 13, wherein the ear-fitting attachment is further configured such that:
a cross-sectional shape of a third portion of the anterior surface of the ear-fitting attachment is substantially curved;
the second portion of the anterior surface transitions into the third portion of the anterior surface; and
the first portion of the anterior surface is distinct from the third portion of the anterior surface.

15. The ear-worn device of claim 11, wherein:
the ear-fitting attachment comprises a first coupling device;
the hooking body comprises a second coupling device; and
the hooking body and the ear-fitting attachment are couplable together by coupling the first coupling device of the ear-fitting attachment with the second coupling device of the hooking body.

16. The ear-worn device of claim 11, wherein, when the hooking body and the ear-fitting attachment are coupled together, a surface of the hooking body is contiguous with a surface of the ear-fitting attachment.

17. (canceled)

18. The ear-worn device of claim 1, wherein, while the ear-worn device is in a closed configuration, the hooking body and an engaging portion of the device body are collectively configured to apply a compressive force to a posterior portion of the ear.

19. The ear-worn device of claim 1, wherein transitioning the ear-worn device from an open configuration to a closed configuration while the ear-worn device is secured to the ear causes the ear pad to move proximate to an interior portion of the ear.

20. The ear-worn device of claim 1, wherein:
the ear pad is comprised of an acoustically transparent material; and
the device body further comprises a speaker system positioned beneath the ear pad and configured to output sound towards a back side of the device body through the ear pad.

21. (canceled)

22. The ear-worn device of claim 1, wherein the device body comprises a device cover positioned towards a front of the device body.

23. The ear-worn device of claim 22, wherein the device cover comprises at least one microphone port positioned at an anterior side of the device body and at least one microphone port positioned at a posterior side of the device body.

24. The ear-worn device of claim 22, wherein:
the device body comprises a touch sensor positioned to a back of the device cover; and
the device cover comprises at least one material configured to allow the touch sensor to detect a touch input received on the device cover.

25. The ear-worn device of claim 1, wherein the device body comprises:
a first microphone positioned near a back side of the device body and near a speaker system;
a second microphone positioned near a front, anterior side of the device body;
a third microphone positioned near a front, anterior side of the device body, wherein the third microphone is positioned closer to a bottom of the device body than the second microphone; and
a fourth microphone positioned near a front, posterior side of the device body.

26. The ear-worn device of claim 25, wherein:
the ear-worn device is configured to capture first sound from at least the third microphone and second sound from the fourth microphone; and
the ear-worn device is configured to perform beam-forming noise cancellation using the first sound and the second sound.

27. The ear-worn device of claim 1, wherein the ear pad comprises a pliable material configured to form at least a partial acoustic seal around an interior portion of a human ear when the ear-worn device is secured to the human ear.

28-57. (canceled)
